(19) United States
(12) Patent Application Publication
(10) Pub. No.: US 2010/0144743 A1
(43) Pub. Date: Jun. 10, 2010

(54) COMPOSITIONS AND METHODS FOR INHIBITION OF TYROSINE KINASES

(75) Inventors: William Bornmann, Missouri City, TX (US); David Maxwell, Pearland, TX (US); Zhenghong Peng, Missouri City, TX (US); Liwei Guo, Birmingham, AL (US)

Correspondence Address: Nielsen IP Law LLC, 1177 West Loop South, Suite 1600, Houston, TX, 77027, US

(73) Assignee: BOARD OF REGENTS, THE UNIVERSITY OF TEXAS SYSTEM, Austin, TX (US)

(21) Appl. No.: 12/439,402

(22) PCT Filed: Sep. 4, 2007
(86) PCT No.: PCT/US2007/077511

§ 371 Date: Feb. 9, 2010

Related U.S. Application Data
(60) Provisional application No. 60/824,501, filed on Sep. 5, 2006.

(51) Int. Cl. A61K 31/506; C07D 239/42; C07D 403/10; C07D 471/04; A61K 31/4985; A61P 35/00

(52) U.S. Cl. 514/249; 544/329; 544/328; 544/350; 514/256

(57) ABSTRACT

Compositions for inhibiting the catalytic activity of tyrosine kinases comprising compounds represented by Formulas (I), (II), and (III). Methods for treating proliferative diseases comprising administering a therapeutically effective amount of the above compositions.

### CROSS-REFERENCE TO RELATED APPLICATIONS

This application claims the benefit of U.S. Provisional Application Ser. No. 60/824,501 filed on Sep. 5, 2006, which is incorporated by reference.

### BACKGROUND

The present disclosure, according to specific example embodiments, generally relates to tyrosine kinases. In particular, the present disclosure relates to compositions useful in inhibiting catalytic activity of tyrosine kinases and associated methods of use.

Tyrosine kinases are a class of enzymes, which catalyze the transfer of the terminal phosphate of adenosine triphosphate to the phenolic hydroxyl group of a tyrosine residue present in the target protein. Tyrosine kinases play a critical role in signal transduction for several cellular functions including cell proliferation, angiogenesis, carcinogenesis, apoptosis, and cell differentiation. Therefore inhibitors of these enzymes would be useful for the treatment or prevention of proliferative diseases which are dependent on these enzymes. Strong epidemiologic evidence suggests that the overexpression or activation of receptor protein tyrosine kinases leading to constitutive mitogenic signaling is an important factor in a growing number of human malignancies, such as cancer. Tyrosine kinases that have been implicated in these processes include Abl, CDK's, EGF, EMT, FGF, FAK, Flk-1/KDR, HER-2, IGF-1R, IR, LCK, MET, PDGF, Src, ephrins, and VEGF.

Ephrin receptors represent the largest family of receptor tyrosine kinases and have emerged as essential regulators of angiogenesis. Angiogenesis refers to the formation of new capillaries from existing vasculature and it is a crucial element in tumor growth. It is required for nourishment and removal of metabolic waste. If neovascularization is insufficient to support the growth and development of tumors, then hypoxia may arise. Tumors with low oxygenation are generally associated with poor prognosis. A key element in the hypoxic response is the hypoxia induced factor (HIF). HIF-1 is a transcription factor that is activated in solid tumors, and is up-regulated along with ephrins and VEGF. As a result of this, ephrins have been described as good targets for anticancer therapies.

### FIGURES

Some specific example embodiments of the disclosure may be understood by referring, in part, to the following description and the accompanying drawings.

FIG. 1 shows a schematic illustrating one of top scoring compounds bound to the ATP binding pocket of ephrin.

FIG. 2 shows a flowchart showing a strategy to analyze the data from in-silico screening.

FIG. 3 shows a synthesis scheme for preparation of one example of a composition of the present disclosure.

FIG. 4 shows a general synthesis scheme for the preparation of one example of a composition of the present disclosure.

FIG. 5 shows a synthesis scheme for LG2-9 and LG2-13 compositions of the present disclosure.

FIG. 6 shows a synthesis scheme for LG2-3 and LG2-7 compositions of the present disclosure.

FIG. 7 shows a synthesis scheme for LG2-11.

FIG. 8 shows a synthesis scheme for LG2-73 and LG2-75.

FIG. 9 shows a synthesis scheme for LG2-87 and LG2-89.

FIG. 10 shows a synthesis scheme for LG2-60 and LG2-65.

FIG. 11 shows a synthesis scheme for LG2-55 and LG2-62.

FIG. 12 shows a synthesis scheme for LG2-85.

FIG. 13 shows a synthesis scheme for LG2-77 and LG2-81.

FIG. 14 shows a synthesis scheme for LG2-95 and LG2-98.

FIG. 15 shows a synthesis scheme for N-(2-hydroxyethyl)-3-(4,4,5,5-tetramethyl-1,3,2-dioxaborolan-2-yl)-benzamide.

FIG. 16 shows a synthesis scheme for LG2-91 and LG2-96.

FIG. 17 shows a synthesis scheme for LG2-101 and LG2-102.

FIG. 18 shows the results of testing for HIF-1 expression (top) and viability (bottom) after exposure of C6#4 cells to PX-478.

FIG. 19 shows the results of testing for HIF-1 expression (top) and viability (bottom) after exposure of C6#4 cells to LG2-7.

FIG. 20 shows the results of testing for HIF-1 expression (top) and viability (bottom) after exposure of C6#4 cells to LG2-11.

FIG. 21 shows the results of testing for HIF-1 expression (top) and viability (bottom) after exposure of C6#4 cells to LG2-13.

FIG. 22 shows the results of testing for HIF-1 expression (top) and viability (bottom) after exposure of C6#4 cells to LG2-62.

FIG. 23 shows the results of testing for HIF-1 expression (top) and viability (bottom) after exposure of C6#4 cells to LG2-65.

FIG. 24 shows the results of testing for HIF-1 expression (top) and viability (bottom) after exposure of C6#4 cells to LG2-75.

FIG. 25 shows the results of testing for HIF-1 expression (top) and viability (bottom) after exposure of C6#4 cells to LG2-81.

FIG. 26 shows the results of testing for HIF-1 expression (top) and viability (bottom) after exposure of C6#4 cells to LG2-85.

FIG. 27 shows the results of testing for HIF-1 expression (top) and viability (bottom) after exposure of C6#4 cells to LG2-89.

FIG. 28 shows the results of testing for HIF-1 expression (top) and viability (bottom) after exposure of C6#4 cells to LG2-96.

FIG. 29 shows the results of testing for HIF-1 expression (top) and viability (bottom) after exposure of C6#4 cells to LG2-98.

FIG. 30 shows the results of testing for HIF-1 expression (top) and viability (bottom) after exposure of C6#4 cells to LG2-102.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure, according to specific example embodiments, generally relates to tyrosine kinases. In particular, the present disclosure relates to compositions useful in inhibiting catalytic activity of tyrosine kinases and associated methods of use.

The compositions of the present disclosure may at least partially inhibit catalytic activity of tyrosine kinases. Examples of tyrosine kinases that may be suitable targets for the compositions of the present disclosure include, but are not limited to, receptor tyrosine kinases and cellular tyrosine kinases. Examples of receptor tyrosine kinases include, but are not limited to, Eph receptors, EGF receptors, insulin receptors, IGF receptor-1, Trk A, PDGF receptors, M-CSF receptors, FGR receptors, VEGF receptors. Examples of cellular tyrosine kinases include, but are not limited to, Src, Frk, Btk, Csk, Abl, ZAP70, Fes, Fps, Fak, Jak, Ack, Yes, Fyn, Lyn, Lck, Blk, Hck, Fgr and Yrk.

The compositions of the present disclosure may at least partially inhibit catalytic activity of a tyrosine kinase directly by interacting with the kinase itself; or indirectly, by interacting with another molecule on which the catalytic activity of the kinase is dependent.

The compositions of the present disclosure may also inhibit HIF-1 expression and HIF-1 signal transduction pathways.

In one embodiment, compositions of the present disclosure may comprise a compound of the following Formula (I):

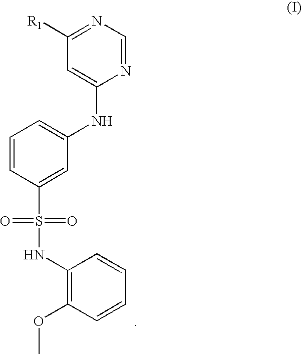

In another embodiment, compositions of the present disclosure may comprise a compound of the following Formula (II):

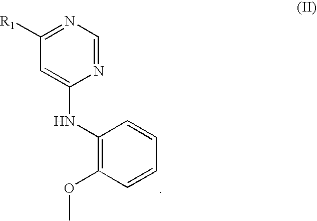

In Formula (I) or in Formula (II), R1 may represent a group selected from the following:

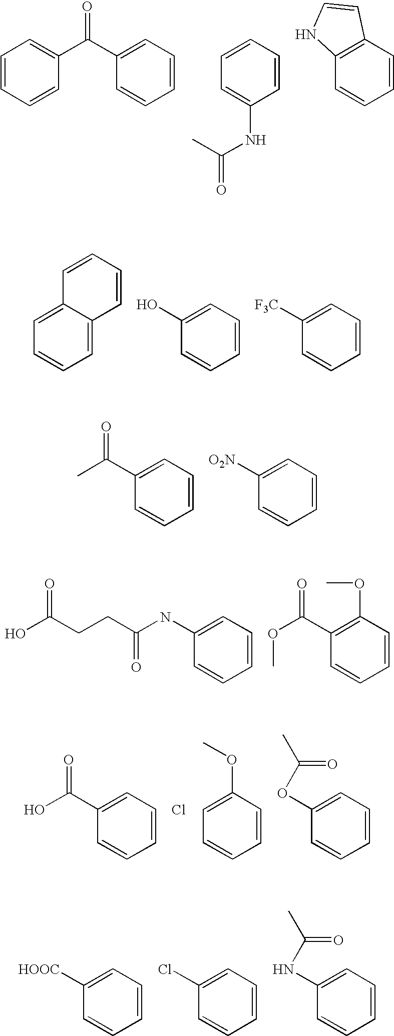

The compounds of Formula (I) and (II) may be enantiomers, diastereomers, pharmaceutically acceptable salts, hydrates, prodrugs, or solvates thereof.

In another embodiment, the compositions of the present disclosure may comprise a compound of the following Formula (III):

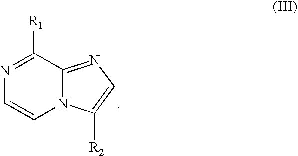

In Formula (III), R₁ may represent a group selected from the following:

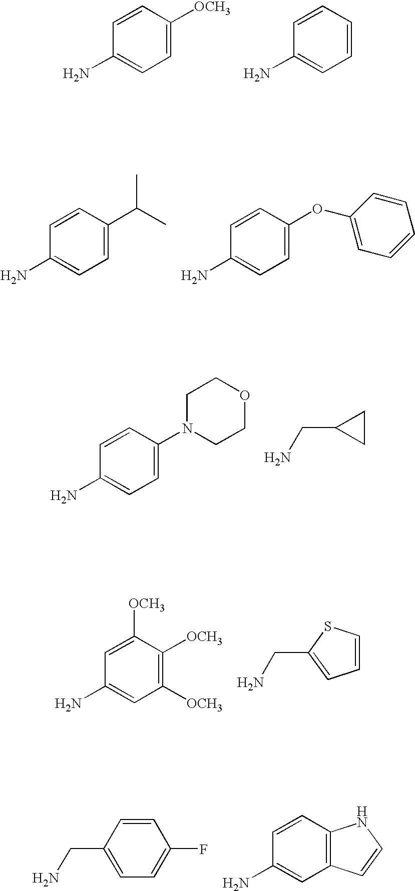

In Formula (III), R₂ may represent a group selected from the following:

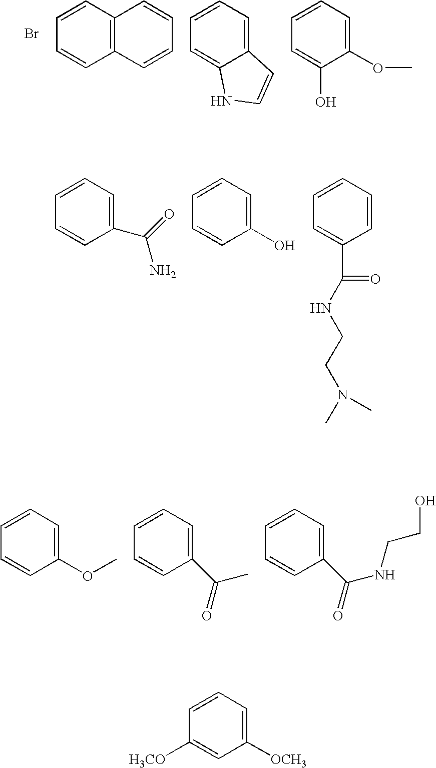

The compounds of Formula (III) may be enantiomers, diastereomers, pharmaceutically acceptable salts, hydrates, prodrugs, or solvates thereof.

The compositions of the present disclosure generally may be synthesized using methods known in the art, including for example, suzuki coupling reactions.

The compositions of the present disclosure also may be provided as a pharmaceutical composition comprising a compound of Formula (I), (II), or (III) and a pharmaceutically acceptable carrier.

The compositions also may be used in a pharmaceutical composition comprising a compound represented by Formula (I), (II), or (III) in combination with pharmaceutically acceptable carrier and an anti-cancer or cytotoxic agent. In certain embodiments, the anti-cancer or cytotoxic agent may be chosen from one or more of linomide; inhibitors of integrin αVβ3 function; angiostatin; razoxane; tamoxifen; toremifene; raloxifene; droloxifene; iodoxifene; megestrol acetate; anastrozole; letrozole; borazole; exemestane; flutamide; nilutamide; bicalutamide; cyproterone acetate; gosereline acetate; leuprolide; finasteride; metalloproteinase inhibitors; inhibitors of urokinase plasminogen activator receptor function; growth factor antibodies; growth factor receptor antibodies such as Avastin® (bevacizumab) and Erbitu® (cetuximab); tyrosine kinase inhibitors; serine/threonine kinase inhibitors; methotrexate; 5-fluorouracil; purine; adenosine analogues; cytosine arabinoside; doxorubicin; daunomycin; epirubicin; idarubicin; mitomycin-C; dactinomycin; mithramycin; cisplatin; carboplatin; nitrogen mustard; melphalan; chlorambucil; busulphan; cyclophosphamide; ifosfamide nitrosoureas; thiotepa; vincristine; Taxol® (pacliatxel); Taxotere® (docetaxel); epothilone analogs; discodermolide analogs; eleutherobin analogs; etoposide; teniposide; amsacrine; topotecan; flavopyridols; biological response modifiers and proteasome inhibitors such as Velcade® (bortezomib).

The compounds represented by Formula (I), (II), or (III) or a pharmaceutically acceptable salt or hydrate or solvate thereof may be administered to a mammal, including a human, to treat cancers of that mammal. The administration method may include, for example, oral or parenteral. As used herein, the term “cancer” refers to an abnormal growth of cells which tend to proliferate in an uncontrolled way, including neoplasms, tumors, and leukemia.

In certain embodiments, the methods of the present disclosure may provide for control of neovascularization of tumor cells using compounds represented by Formula (I), (II), or (III). In these embodiments, the compounds represented by Formula (I), (II), or (III) may bind to a tyrosine kinase, thereby inhibiting its activity. In some embodiments, the angiogenesis of tumor cells may depend on the activation of tyrosine kinases, and thus, inhibition of tyrosine kinases may decrease tumor growth and development.

In certain embodiments, a compound represented by Formula (I), (II), or (III) or a pharmaceutically acceptable salt or hydrate or solvate thereof may be used to inhibit catalytic activity of tyrosine kinases in cancer cells in a dose dependent manner. It will be recognized by one of skill in the art that the optimal quantity and spacing of individual dosages of a compound represented by Formula (I), (II), or (III) will be determined by the nature and extent of the condition being treated, the form, route and site of administration, and the particular patient being treated, and that such optimums can be determined by conventional techniques. Similarly, the optimal course of treatment, for example, the number of doses of a compound represented by Formula (I), (II), or (III) given per day for a defined number of days, can be ascertained by those skilled in the art using conventional course of treatment determination tests.

The present disclosure also provides methods of inhibiting protein kinase activity of tyrosine kinases which may comprise administering to a mammal in need thereof, a therapeutically effective amount of a compound of Formula (I), (II), or (III). As used herein, the term “therapeutically effective amount” refers to the amount of an active compound or pharmaceutical agent that elicits the desired biological or medicinal response in a tissue, system, animal or human. In certain embodiments, the methods of the present disclosure may be useful in inhibiting tyrosine kinases of the Eph family of receptors.

In certain embodiments, the compositions of the present disclosure may be used to treat a proliferative disease, comprising administering to a mammal in need thereof, a therapeutically effective amount of a compound of Formula (I), (II), or (III). In certain embodiments, the proliferative disease may be cancer.

To facilitate a better understanding of the present invention, the following examples of specific embodiments are given. In no way should the following examples be read to limit or define the entire scope of the invention.

### EXAMPLES

The design, synthesis, and preliminary results from the in-vitro testing of compounds designed as inhibitors of ephrin kinase, specifically ephrin A4 are hereinafter described. The design resulted from the in-silico screening of several targeted libraries via docking to a homology model of ephrin A4 constructed from the nearly identical ephrin A2 crystal structure, followed by post-filtering using several criteria. This filtering included consideration of the binding mode, docking score, and consensus docking score. This resulted in several structural cores and two of those cores were selected based on synthetic feasibility. Several compounds were synthesized based on the core and structures identified by in-silico screening. These were tested in an ephrin A4 kinase assay and found to have only marginal activity at the 10 uM level. Knowledge of a link between hypoxia induced factor-1 alpha (hif1alpha) and ephrin, led to the same compounds and additional analogs being tested in an assay used to gauge hif1alpha pathway inhibition. In these assays, several compounds were found to inhibit hif1alpha expression in the low uM range that was separate from a much higher general cytotoxicity value.

Structure-Based Design of Ephrin Inhibitors.

Since no crystal structure currently exists for ephA4, the crystal structure of ephA2 (PDB accession code 1MQB) was used as a template to construct a homology model. The primary amino acid sequence for human ephA4 was located with the tools available from NCBI (http://www.ncbi.nlm.nih.gov), accession code NP_{—}004429. The sequence was truncated to 615-881 and this region was read in as a raw sequence to Swiss-PDB Viewer. A search for suitable templates, not surprisingly, led to ephB2 (1JPA) and the aforementioned ephA2 structure. A fit was completed to initially align the sequences and prepare for model building. Due to the high sequence identity, the alignment did not require any further modification; however, there was a missing structural segment corresponding to the activation loop, which is noted in the paper discussing the crystal structure. This alignment was submitted to the Swiss-Model server and it returned a viable structure to begin design work. Although the sequence is nearly identical, it was not until the homology model was constructed that the extent of differences in the ATP binding site region could be assessed. The crystal structure of ephA2 and the homology model of ephA4 were compared in the region within 5 Å of the ANP ligand. In this region, the sequence identity between these two receptors is nearly 100%, with only two residues differing between them. Several commercially available kinase targeted libraries were prepared for in-silico screening and the docking of these libraries led to the identification of several functionalized cores to use as “lead” structures.

The design of ephrin inhibitors provided herein includes the selection of screening compounds, in-silico screening via docking, analysis of docking results, preliminary selection of compounds with a degree of specificity toward ephrin A4 kinase, and final selection of candidate compounds.

Compounds from three vendors: Asinex (Winston-Salem, N.C.), BioFocus (Saffron Walden, Essex, UK), and LifeChem (Burlington, ON, Canada) were screened in-silico against ephrin A4 via docking of the individual ligands into the receptor binding site.

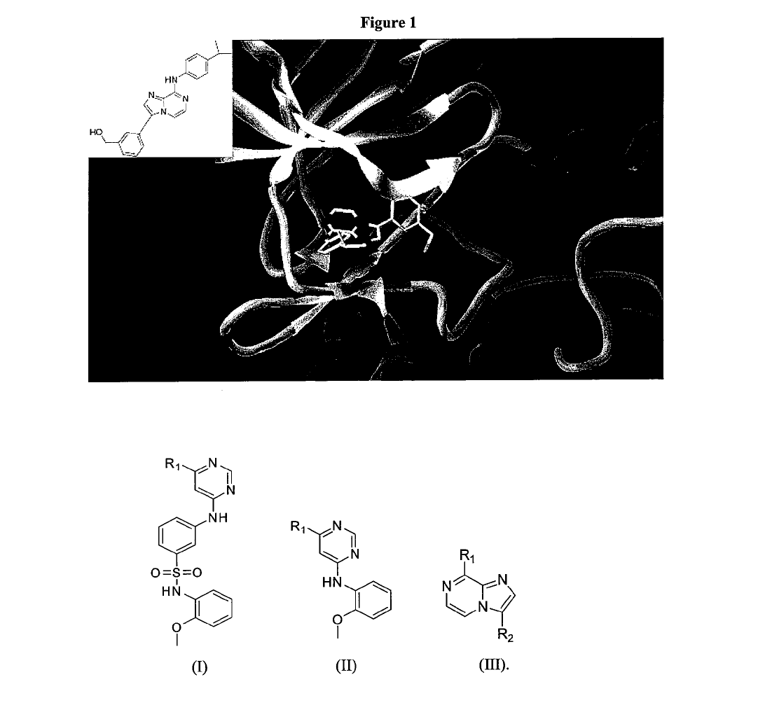

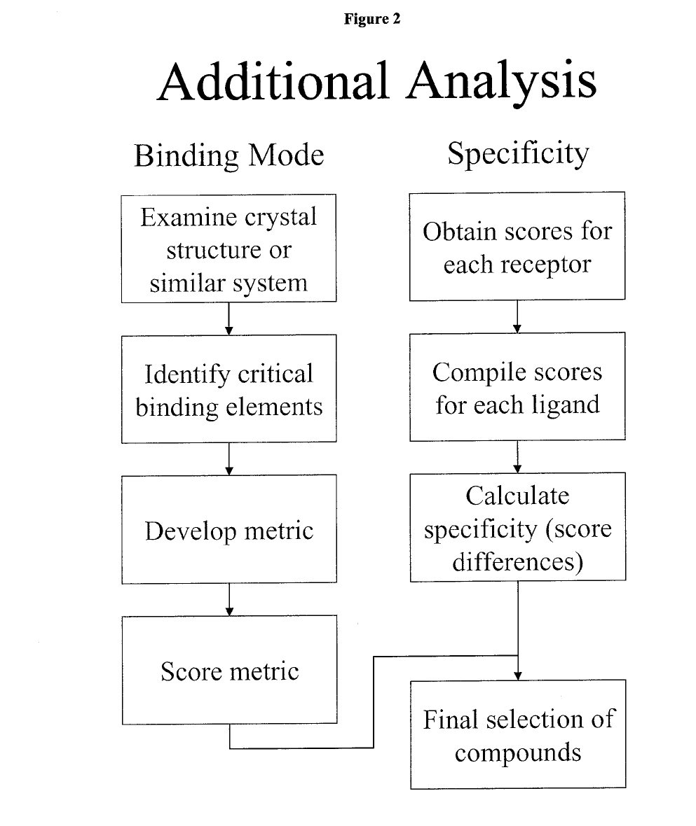

The traditional metric for this type of screening has been the individual docking scores, which aim to capture the binding affinity of the ligand and receptor. Though useful in ranking ligands, the scoring methods are imperfect, so the consensus of several scoring methods (Clark, et al., (2002) J Mol Graph Model 20, 281-95) is generally utilized in the final selection. In the present study, the consensus scoring was completed with the scoring methods provided in the FlexX module within Sybyl 7.1. Over 32,000 compounds were screened in this manner. While compounds with the highest scores were of interest, additional metrics were also considered. One additional element used in the selection of compounds was the evaluation of binding mode. Utilization of specific interactions along with the docking scores has been shown to increase the hit ratio of in-silico screening. Information on these binding interactions was derived from the inhibitor bound crystal structure. An example of such a structure is provided by FIG. 1 which is a schematic showing one of top scoring compounds bound to the ATP binding pocket of ephrin. This level of analysis was not available as part of the normal docking interface, so code was written to analyze the data and provide consensus information. A flowchart showing this strategy is shown in FIG. 2. The analysis included identifying hydrogen bonding elements within each docking configuration for each ligand and comparing those against the specific areas of the receptor site. Interactions with the NH hydrogen of Met88 and Carbonyl oxygen of Met88 were mandated and those compounds within a particular distance were flagged and the combination of elements was used as a filter for the binding mode. This procedure was followed for docked configurations determined for each of the compounds in the screening libraries. Several candidate compounds were identified in this manner. After restricting to a particular binding mode, the above mentioned filters were then applied in the order of docking score then consensus score. This computational strategy indicated that compounds based on a Imidazo[1,2-a]pyrazine and Imidogen, 4-pyrimidinyl-cores may be effective for inhibition of ephrin kinase.

Preparation of Ephrin Inhibitors.

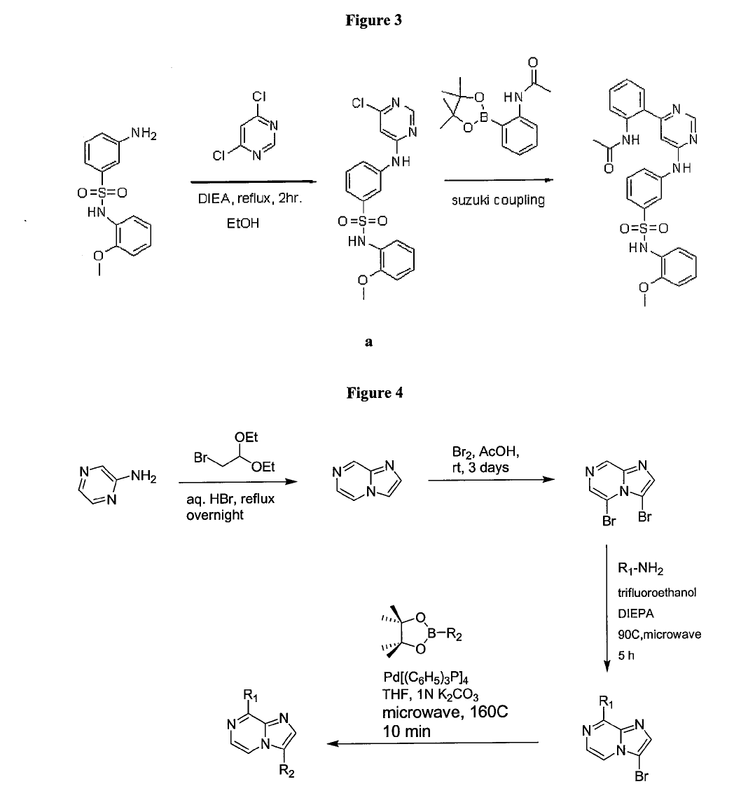

FIG. 3 illustrates a synthesis scheme for preparation of one example of a composition of the present disclosure. 4,6-dichloropyrimidine (0.59 g, 4 mmol) is dissolved with 2 g of N-(2-methoxyphenyl)-3-aminobenzenesulfonamide (4 mmol) in 15 ml ethanol, then added 1.75 ml DIEA (10 mmol). Reaction in under reflux for 2 hr and cooled down to ambient temperature. After evaporating solvent, the crude product was purified by flash chromatography with a linear gradient of hexane and ethyl acetate to give N-(2-methoxyphenyl)-3-(6-Chloro-pyrimidine-4-yl)-benzenesulfonamide (a in FIG. 3). Yield 1 g. (71%). MS: 391.1 (M+H);

50 mg (0.13 mmol) N-(2-methoxyphenyl)-3-(6-Chloro-pyrimidine-4-yl)-benzenesulfonamide, 2′-(4,4,5,5-Tetramethyl-1,3,2-dioxaborolan-2-yl)acetanilide (0.13 mmol), 70 mg (0.5 mmol) of potassium carbonate and 10 mg palladium tetrakis triphenylphonsphine (0.01 mmol) was mixed in MeCN/water (1:1) 4 ml. After fill with argon, the reaction was performed under microwave at 150° C. for 30 min. Filter hot reaction solution was filtered and the product (b in FIG. 3) was crystallized from the reaction mixture. The pale solid is collected by filtration and washed with water. 50 mg (80%). MS: 490.5 (M+H); ¹H NMR (DMSO) δ 11.14 (s, 1H), 10.01 (s, 1H), 8.78 (s, 1H), 8.15 (s, 1H), 8.10 (d, J=8.0 Hz, 1H), 7.67 (t, J=8.0 Hz, 1H), 7.48 (m, 2H), 7.26 (d, J=8.0 Hz, 1H), 7.26 (m, 2H), 7.09 (s, 2H), 6.87 (m, 3H), 3.5 (s, 3H), 2.07 (s, 3H). Additional examples of synthesized inhibitors and their corresponding properties are described in Table 1 below.

TABLE 1

| Formula | Structure | Properties |
|---|---|---|
| C₂₃H₂₀N₄O₄S | 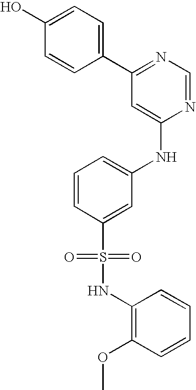 | Molecular Weight: 448.49 MS: 449.4 (M + H); ¹H NMR(DMSO) δ 11.41 (s, 1H), 9.58 (s, 1H), 8.83 (s, 1H), 8.12 (s, 1H), 7.91 (d, , J = 8.0 Hz, 1H), 7.83 (d, , J = 8.0 Hz, 2H), 7.60 (m, 4H), 7.22 (d, 1H), 7.15 (m, 1H), 7.00 (d, 1H), 6.90 (m, 2H), 3.5 (s, 3H). |
| C₂₃H₁₉N₅O₅S | 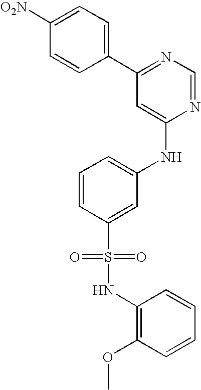 | Molecular Weight: 477.49 MS: 478.3 (M + H); |
| C₂₄H₂₀N₄O₅S | 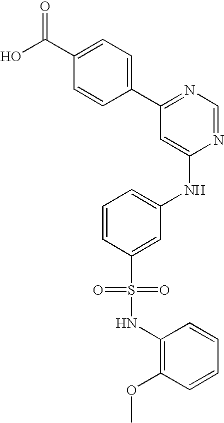 | Molecular Weight: 476.5 MS: 477.6 (M + H); |
| C₂₅H₂₂N₄O₅S | 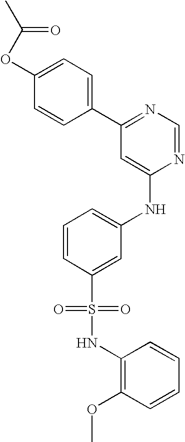 | Molecular Weight: 490.53 MS: 449.5 (M + H − Ac). |
| C₂₅H₂₃N₅O₄S | 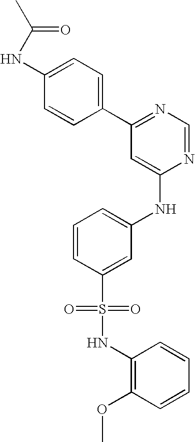 | Molecular Weight: 489.55 MS: 490.4 (M + H); ¹H NMR(DMSO) δ 10.21 (s, 1H), 9.95 (s, 1H), 8.68 (s, 1H), 8.17 (s, 1H), 7.99 (d, J = 8.4 Hz, 2H), 7.89 (d, J = 8.4 Hz, 1H), 7.67 (t, J = 8.0 Hz, 1H), 7.73 (d, J = 8.4 Hz, 2H), 7.45 (t, J = 7.8 Hz, 1H), 7.33 (d, J = 7.8 Hz, 1H), 7.20 (m, 2H), 7.10 (s, 1H), 6.83 (m, 2H), 3.5 (s, 3H), 2.97 (s, 3H). |
| C₂₄H₁₉F₃N₄O₃S | 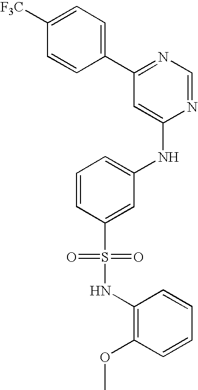 | Molecular Weight: 500.49 MS: 501.3 (M + H); ¹H NMR(DMSO) δ 11.14 (s, 1H), 8.78 (s, 1H), 8.24 (d, J = 8.4 Hz, 2H), 8.14 (s, 1H), 7.93 (m, 3H), 7.46 (t, J = 8.0 Hz, 1H), 7.36 (d, J = 8.0 Hz, 1H), 7.33 (s, 1H), 7.19 (d, J = 8.0 Hz, 1H), 6.90 (s, 1H), 6.85 (m, 2H), 6.70 (s, 1H), 3.53 (s, 3H). |
| C₃₀H₂₄N₄O₄S | 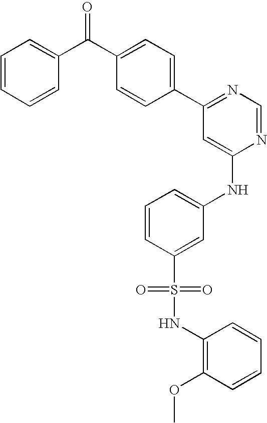 | Molecular Weight: 536.6 MS: 537.4 (M + H); ¹H NMR(DMSO) δ 10.09 (s, 2H), 8.78 (s, 1H), 8.22 (d, J = 8.4 Hz, 2H), 8.18 (s, 1H), 7.93 (d, 1H), 7.90 (d, J = 8.4 Hz, 2H), 7.72 (t, 1H), 7.61 (t, J = 8.0 Hz, 2H), 7.49 (t, 1H), 7.40 (d, 1H), 7.34 (s, 1H), 7.24 (d, 1H), 7.09 (s, 1H), 6.87 (m, 3H), 3.5 (s, 3H). |
| C₂₇H₂₂N₄O₃S | 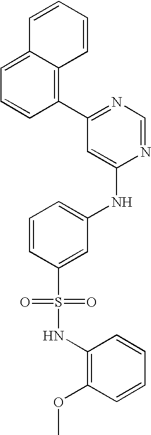 | Molecular Weight: 482.55 MS: 483.4 (M + H); |
| C₂₅H₂₂N₄O₄S | 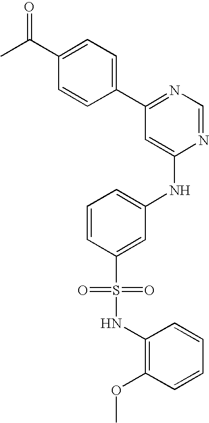 | Molecular Weight: 474.53 ¹H NMR(DMSO) δ 11.14 (s, 1H), 10.05 (s, 1H), 8.60 (s, 1H), 8.29 (s, 1H), 8.19 (s, 1H), 8.12 (d, , J = 8.0 Hz, 1H), 7.91 (d, , J = 8.0 Hz, 1H), 7.71 (t, J = 8.0 Hz, 1H), 7.46 (t, J = 8.0 Hz, 1H), 7.36 (m, 2H), 7.21 (d, J = 8.0 Hz, 1H), 7.05 (t, 1H), 6.86 (d, J = 8.0 Hz, 1H), 6.81 (t, 1H), 6.69 (br, 1H), 3.92 (s, 3H), 1.77 (s, 3H). |
| C₂₆H₂₄N₄O₆S | 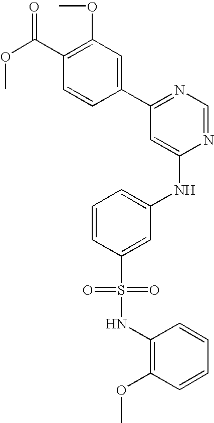 | Molecular Weight: 520.56 MS: 507.4 (M + H − Me); ¹H NMR(DMSO) δ 10.20 (s, 1H), 9.48 (s, 1H), 8.78 (s, 1H), 8.22 (s, 1H), 8.10 (d, J = 8.0 Hz, 1H), 7.91 (d, 1H), 7.79 (d, 1H), 7.76 (s, 1H), 7.63 (t, J = 8.0 Hz, 1H), 7.50 (t, 1H), 7.33 (s, 1H), 7.25 (d, , 2H), 7.12 (t, 1H), 6.87 (m, 3H), 6.70 (vr, 1H), 3.92 (s, 3H), 3.50 (s, 6H). |
| C₂₄H₂₂N₄O₄S | 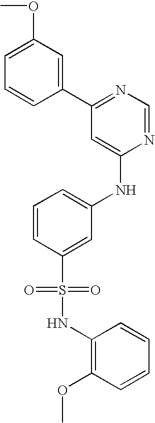 | MS: 463.3 (M + H); ¹H NMR(DMSO) δ 9.98 (s, 1H), 8.72 (s, 1H), 8.19 (s, 1H), 7.92 (d, 1H), 7.59 (m, 2H), 7.46 (dd, 2H), 7.35 (d, 1H), 7.26 (m, 2H), 7.10 (m, 2H), 6.87 (m, 2H), 3.85 (s, 3H), 3.50 (s, 3H). |
| C₂₃H₁₉ClN₄O₃S | 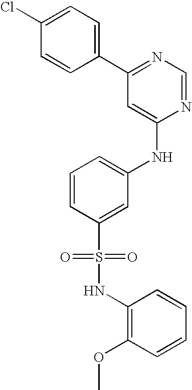 | Molecular Weight: 466.94 MS: 467.3 (M + H); |
| C₂₅H₂₁N₅O₃S | 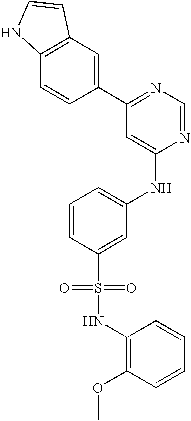 | Molecular Weight: 471.53 MS: 472.4 (M + H); ¹H NMR(DMSO) δ 11.86 (s, 1H), 11.68 (s, 1H), 9.60 (s, 1H), 8.30 (s, 1H), 8.17 (s, 1H), 7.98 (d, , J = 8.0 Hz, 1H), 7.8-7.5 (m, 5H), 7.25 (d, 1H), 7.13 (t, 1H), 7.42 (t, 1H), 6.95 (m, 2H), 6.70 (m, 2H), 3.5 (s, 3H). |
| C₂₇H₂₄N₅O₆S | 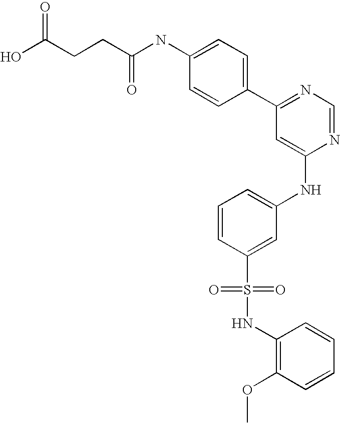 | Molecular Weight: 546.57 MS: 548.3 (M + H); ¹H NMR(DMSO) δ 11.24 (s, 1H), 10.01 (s, 1H), 8.67 (s, 1H), 8.20 (s, 1H), 7.99 (d, , J = 8.0 Hz, 2H), 7.89 (d, , J = 8.0 Hz, 1H), 7.71 (d, , J = 8.0 Hz, 2H), 7.66 (m, 1H), 7.42 (t, 1H), 7.30 (d, 1H), 7.21 (d, 2H), 7.05 (t, 1H), 6.80 (s, 3H), 6.70 (m, 2H), 3.5 (s, 3H), 2.64 (t, 2H), 2.58 (t, 2H). |
| C₁₁H₁₀ClO | 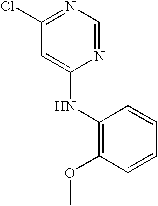 | ¹H NMR(DMSO) δ 9.54 (s, 1H), 9.28 (s, 1H), 8.37 (s, 1H), 7.77 (br, 1H), 7.67 (br, 2H), 7.15 (t, J = 7.2 Hz, 1H), 7.08 (d, J = 8.4 Hz, 1H), 6.95 (t, J = 7.2 Hz, 1H), 3.85 (s, 3H). |
| C₁₉H₁₈N₄O₂ | 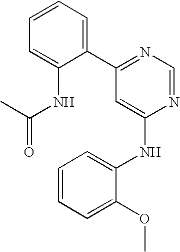 | Molecular Weight: 334.37 |
| C₁₇H₁₅N₃O₂ | 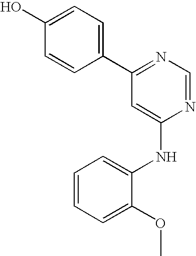 | Molecular Weight: 293.32 MS: 293.9 (M + H); ¹H NMR(DMSO) δ 10.50 (s, 2H), 8.76 (s, 1H), 7.79 (d, J = 8.4 Hz, 1H), 7.67 (br, 2H), 7.30 (t, J = 7.2 Hz, 1H), 7.18 (d, J = 8.4 Hz, 1H), 7.03 (t, J = 8.4 Hz, 1H), 6.99 (d, J = 8.4 Hz, 1H), 3.85 (s, 3H). |
| C₁₇H₁₄N₄O₃ | 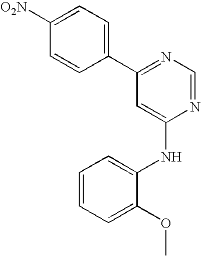 | Molecular Weight: 322.32 MS: 322.4 (M + H) |
| C₁₉H₁₇N₃O₃ | 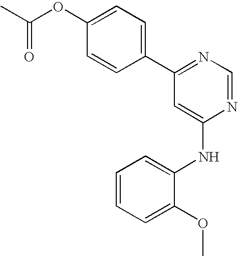 | Molecular Weight: 335.36 |
| C₁₉H₁₈N₄O₂ | 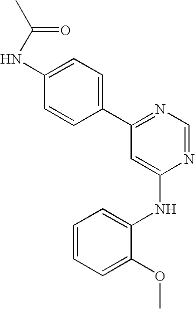 | Molecular Weight: 334.37 MS: 335.5 (M + H); ¹H NMR(DMSO) δ 10.75 (s, 1H), 10.53 (s, 2H), 8.80 (s, 1H), 7.89 (d, J = 8.4 Hz, 2H), 7.85 (d, J = 8.4 Hz, 2H), 7.64 (br, 1H), 7.33 (t, J = 7.2 Hz, 1H), 7.18 (d, J = 8.4 Hz, 1H), 7.03 (t, J = 8.4 Hz, 1H), 3.83 (s, 3H), 2.11 (s, 3H). |
| C₁₈H₁₄F₃N₃O | 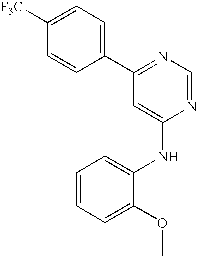 | Molecular Weight: 345.32 MS: 347.0 (M + H); ¹H NMR(DMSO) δ 10.01 (s, 1H), 8.81 (s, 1H), 8.16 (d, J = 8.4 Hz, 2H), 7.97 (d, J = 8.4 Hz, 2H), 7.84 (br, 1H), 7.24 (t, J = 7.2 Hz, 1H), 7.16 (d, J = 8.4 Hz, 1H), 7.0 (t, J = 8.4 Hz, 1H), 3.85 (s, 3H). |
| C₂₄H₁₉N₃O₂ | 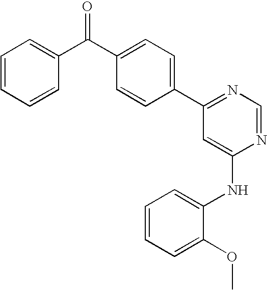 | Molecular Weight: 381.43 |
| C₂₁H₁₇N₃O | 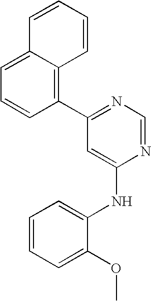 | Molecular Weight: 327.38 |
| C₁₉H₁₇N₃O₂ | 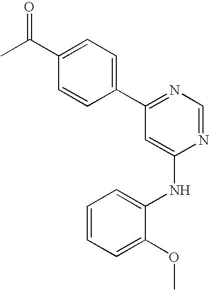 | Molecular Weight: 319.36 MS: 320.0 (M + H); ¹H NMR(DMSO) δ 10.12 (s, 1H), 8.83 (s, 1H), 8.24 (s, 1H), 7.79 (d, J = 8.4 Hz, 1H), 7.67 (br, 1H), 7.30 (t, J = 7.2 Hz, 1H), 7.18 (d, J = 8.4 Hz, 1H), 7.03 (t, J = 8.4 Hz, 1H), 6.99 (d, J = 8.4 Hz, 1H), 3.85 (s, 3H). |
| C₂₀H₁₉N₃O₄ | 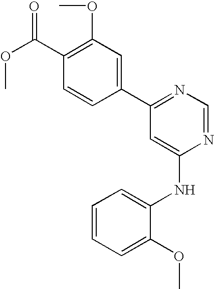 | Molecular Weight: 365.38 MS: 352.3 (M + 2H − Me); |
| C₁₈H₁₇N₃O₂ | 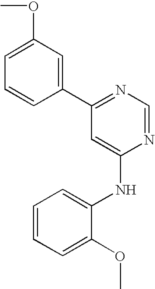 | Mol. Wt.: 307.35 MS: 308.8 (M + H); |
| C₁₈H₁₅N₃O₃ | 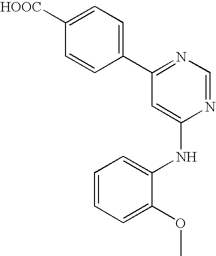 | Molecular Weight: 321.33 MS: 322.4 (M + H); ¹H NMR(DMSO) δ 10.24 (s, 1H), 8.82 (s, 1H), 8.13 (d, J = 8.4 Hz, 2H), 8.05 (d, J = 8.4 Hz, 2H), 7.79 (br, 1H), 7.26 (br, 1H), 7.17 (d, J = 8.4 Hz, 1H), 7.03 (t, J = 8.4 Hz, 1H), 3.85 (s, 3H). |
| C₁₇H₁₄ClN₃O | 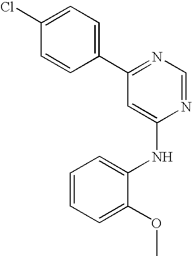 | Molecular Weight: 311.77 |
| C₁₉H₁₆N₄O | 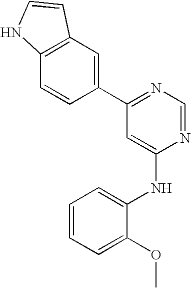 | Molecular Weight: 316.36 MS: 317.5 (M + H) |
| C₂₁H₁₉N₄O₄ | 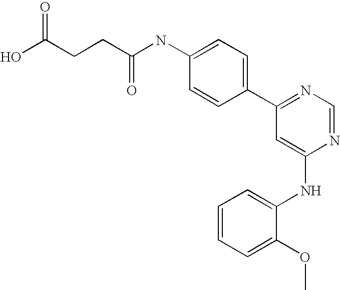 | Molecular Weight: 391.4 MS: 393.4 (M + H); ¹H NMR(DMSO) δ 10.69 (s, 1H), 10.50 (br, 1H), 8.80 (s, 1H), 7.89 (d, J = 8.4 Hz, 2H), 7.85 (d, J = 8.4 Hz, 2H), 7.60 (br, 1H), 7.37 (t, J = 7.2 Hz, 1H), 7.18 (d, J = 8.4 Hz, 1H), 7.03 (t, J = 8.4 Hz, 1H), 3.85 (s, 3H), 2.64 (t, 2H), 2.58 (t, 2H). |

FIG. 4 illustrates a general synthesis scheme for the preparation of one example of a composition of the present disclosure. Examples of these compositions are hereinafter classified as LG2-#. All commercial reagents were used as received. 1H and ¹³C NMR and 2D-NMR spectra were recorded at ambient temperature using a 600 MHz Bruker Ultrashield™plus spectrometer. The chemical shifts are reported in 8 values (ppm) relative to an internal reference of tetramethylsilane (TMS). Mass spectra were obtained from Applied Biosystems QTRAP LC/MS/MS system (electrospray, positive mode). All reactions were carried out in dry glassware and were protected from atmospheric moisture. Thin-layer chromatography (TLC) was performed on a Merck TLC aluminum sheet (silica gel 60 F254). Preparative separations were performed on RediSep™ flash columns under ISCO CombiFlash® Companion™ system. Microwaved synthesis was performed on CEM ExplorerPLS® system, and Discover® platform.

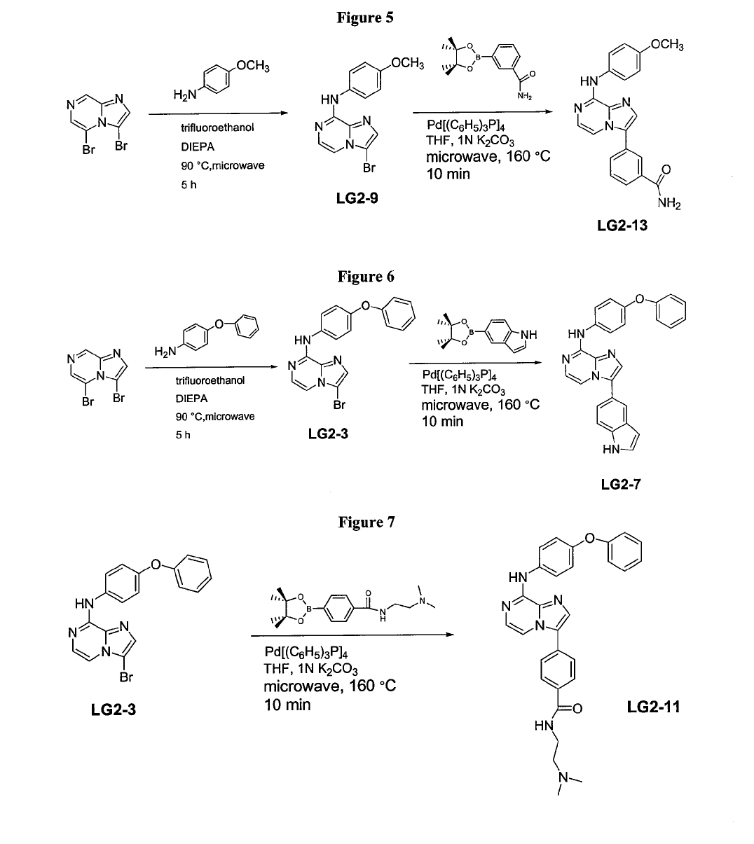

FIG. 5 illustrates a synthesis scheme for LG2-9 and LG2-13. To prepare LG2-9, a solution of 3,5-dibromo-imidazo[1,2-a]pyrazine (2.02 g, 7.29 mmol), p-anisidine (4.30 g, 34.91 mmol), and N,N-diisopropylethylamine (6.6 ml, 37.88 mmol) in CF3CH2OH (25 ml) was heated at 90° C. under microwave irradiation for 5 hours. The volatiles were evaporated, and the residue (re-dissolved in CH2Cl2) was subjected to flash column chromatography on silica gel (elution with hexane-EtOAc) to give LG2-9 as a yellow solid (549 mg, 23%). ESI-MS: m/z 319 (M+1), 321 (M+2+1); 111 NMR (600 MHz, CDCl3): δ 7.79 (1H, br. s, NH), 7.70 (2H, d, J=9.0 Hz), 7.53 (2H, d+s, J=4.2 Hz), 7.50 (1H, d, J=4.2 Hz), 6.93 (2H, d, J=9.0 Hz), 3.82 (3H, s, OCH3); 13C NMR (150.9 MHz, CDCl₃): δ 155.99, 146.41, 133.60, 132.22, 131.89, 129.23, 121.88, 114.36, 108.82, 98.10, 55.56.

For LG2-13 synthesis, to a solution of LG2-9 (98 mg, 0.307 mmol), 3-(4,4,5,5-tetramethyl-1,3,2-dioxaborolan-2-yl)-benzamide (116 mg, 0.469 mmol, 1.5 equiv.), and Tetrakis(triphenylphosphine) palladium(0) [Pd(PPh3)4, 41 mg, 0.035 mmol) in THF (2.0 ml) was added 1.0 ml of aqueous 1 N K2CO3 solution. The resultant mixture was heated at 160° C. under microwave irradiation for 10 min. The mixture was diluted with EtOAc (15 ml), and the organic phase was separated out. After removal of solvent, the residue was subjected to flash column chromatography on silica gel (elution with CH2Cl2-MeOH) to give LG2-13 as a white solid (67 mg, 61%). ESI-MS: m/z 360 (M+1); ¹H NMR (600 MHz, MeOH-d4): δ 8.14 (1H, s), 7.97 (1H, d, J=7.8 Hz), 7.89 (1H, d, J=4.8 Hz), 7.84 (1H, d, J=7.8 Hz), 7.77 (1H, s), 7.72 (2H, d, J=6.6 Hz), 7.68 (1H, t, J=7.8 Hz), 7.42 (1H, d, J=4.8 Hz), 6.95 (2H, d, J=6.6 Hz), 3.81 (3H, s, OCH3); 13C NMR (150.9 MHz, CDCl3-MeOH-d4 85:15 v/v): δ 169.59, 155.80, 146.78, 134.51, 133.57, 132.09, 131.15, 130.65, 129.50, 128.92, 128.66, 127.66, 127.55, 127.23, 121.95, 114.21, 108.62, 55.43.

FIG. 6 illustrates a synthesis scheme for LG2-3 and LG2-7. To prepare LG2-3, a solution of 3,5-dibromo-imidazo[1,2-a]pyrazine (318 mg, 1.0 mmol), 4-phenoxyaniline (0.97 g, 5.2 mmol), and N,N-diisopropylethylamine (0.95 ml, 5.4 mmol) in CF3CH2OH (5.4 ml) was heated at 90° C. under microwave irradiation for 5 hours. The volatiles were evaporated, and the residue (re-dissolved in CH2Cl2) was subjected to flash column chromatography on silica gel (elution with hexane-EtOAc) to yield LG2-3 as a pale brown solid (70 mg, 18%). ESI-MS: m/z 381 (M+1), 383 (M+2+1); ¹H NMR (600 MHz, CDCl₃): δ 7.87 (1H, br. s), 7.80 (2H, d, J=9.0 Hz), 7.56 (1H, d, J=4.8 Hz), 7.55 (1H, s), 7.54 (1H, d, J=4.8 Hz), 7.32 (2H, m), 7.09-7.01 (5H, m); 13C NMR (150.9 MHz, CDCl₃): δ 157.81, 152.68, 146.12, 134.53, 133.75, 132.32, 129.70, 129.08, 122.93, 121.39, 119.89, 118.35, 109.15, 98.23.

For synthesis of LG2-7, to a solution of LG2-3 (70 mg, 0.183 mmol), 5-(4,4,5,5-tetramethyl-1,3,2-dioxaborolan-2-yl)-1H-indole (67 mg, 0.275 mmol, 1.5 equiv.), and Tetrakis(triphenylphosphine)-palladium(0) (25 mg, 0.022 mmol) in THF (2.0 ml) was added 1.0 ml of aqueous 1 N K2CO3 solution. The resultant mixture was heated at 160° C. under microwave irradiation for 10 min. The mixture was diluted with EtOAc (15 ml), and the organic phase was separated out. After removal of solvent, the residue was subjected to flash column chromatography on silica gel (elution with CH2Cl2-MeOH) to yield LG2-7 as a white solid (46 mg, 61%). ESI-MS: m/z 418 (M+1); ¹H NMR (600 MHz, CDCl₃): δ 8.33 (1H, s), 7.99 (1H, s), 7.84 (3H, m), 7.76 (1H, d, J=4.8 Hz), 7.63 (1H, s), 7.56 (1H, d, J=7.8 Hz), 7.45 (1H, d, J=4.8 Hz), 7.38 (1H, dd, J=2.4, 8.4 Hz), 7.34-7.31 (3H, m), 7.09-7.06 (3H, m), 7.03 (2H, m), 6.66 (1H, m); ¹³C NMR (150.9 MHz, CDCl3): δ 152.28, 146.66, 135.82, 135.15, 130.48, 130.09, 129.66, 128.44, 128.22, 125.49, 122.79, 122.50, 121.23, 120.91, 120.00, 119.89, 118.23, 111.90, 109.48, 103.13.

FIG. 7 illustrates a synthesis scheme for LG2-11. To a solution of LG2-3 (80 mg, 0.209 mmol), N-(2-dimethylaminoethyl)-4-(4,4,5,5-tetramethyl-1,3,2-dioxaborolan-2-yl)-benzamide (100 mg, 0.314 mmol, 1.5 equiv.), and Tetrakis(triphenylphosphine)-palladium(0) (28 mg, 0.024 mmol) in THF (2.0 ml) was added 1.0 ml of aqueous 1 N K2CO3 solution. The resultant mixture was heated at 160° C. under microwave irradiation for 10 min. The mixture was diluted with EtOAc (15 ml), and the organic phase was separated out. After removal of solvent, the residue was subjected to flash column chromatography on silica gel (elution with CH₂Cl₂-MeOH) to furnish LG2-11 as a pale yellow foam (85 mg, 82%). ESI-MS: m/z 493 (M+1); ¹H NMR (600 MHz, CDCl₃): δ 8.13 (1H, br. s, NH), 7.98 (2H, d, J=8.4 Hz), 7.82 (2H, d, J=9.0 Hz), 7.70 (1H, d, J=4.8 Hz), 7.67 (1H, s), 7.64 (2H, d, J=8.4 Hz), 7.49 (1H, d, J=4.8 Hz), 7.31 (2H, m), 7.12 (1H, br. s, NH), 7.08-7.00 (5H, m), 3.58 (211, dd, J=10.8, 5.4 Hz), 2.58 (2H, t, J=6.0 Hz), 2.32 (6H, s); ¹³C NMR (150.9 MHz, CDCl₃): δ 166.63, 157.87, 152.50, 146.77, 134.84, 134.55, 134.04, 131.43, 131.36, 129.69, 129.00, 128.13, 127.85, 127.79, 122.88, 121.34, 119.94, 118.28, 109.07, 57.84, 45.14, 37.19.

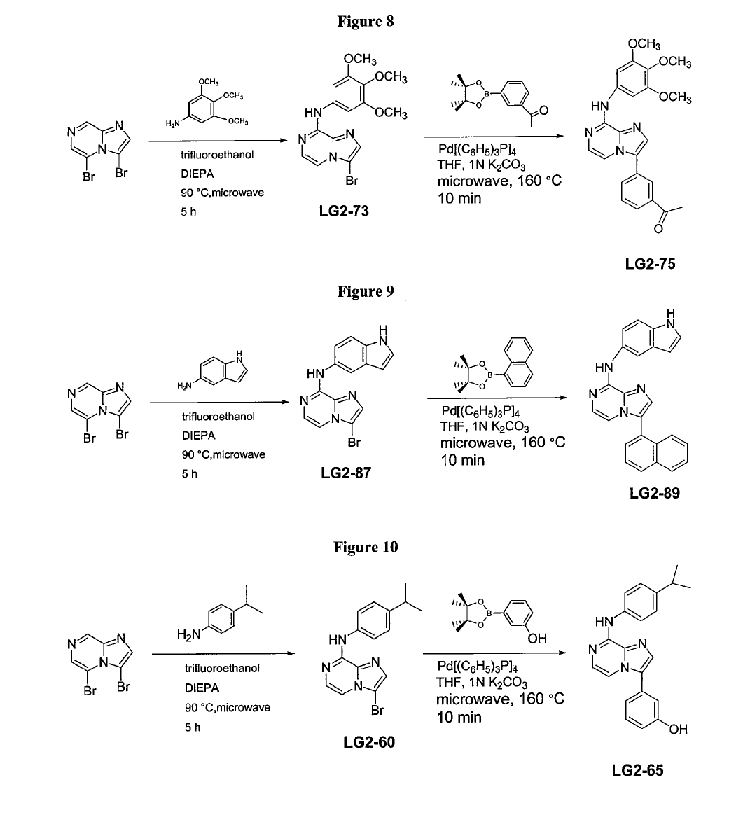

FIG. 8 illustrates a synthesis scheme for LG2-73 and LG2-75. To prepare LG2-73, a solution of 3,5-dibromo-imidazo[1,2-a]pyrazine (512 mg, 1.84 mmol), 3,4,5-trimethoxyaniline (1.66 g, 9.1 mmol), and N,N-diisopropylethylamine (1.65 ml, 9.5 mmol) in CF3CH2OH (6.5 ml) was heated at 90° C. under microwave irradiation for 5 hours. The volatiles were evaporated, and the residue was subjected to flash column chromatography on silica gel (elution with hexane-EtOAc) to yield LG2-73 as a pale yellow foam (126 mg, 18%). ESI-MS: m/z 379 (M+1), 381 (M+2+1); ¹H NMR (600 MHz, CDCl₃): δ 7.89 (1H, s, NH), 7.61 (1H, d, J=4.2 Hz), 7.58 (1H, s), 7.57 (1H d, J=4.2 Hz), 7.19 (2H, s), 3.94 (6H, s), 3.87 (3H, s).

For preparation of LG2-75, to a solution of LG2-73 (30 mg, 0.079 mmol), 143-(4,4,5,5-tetramethyl-1,3,2-dioxaborolan-2-yl)phenylkethanone (29 mg, 0.118 mmol, 1.5 equiv.), and Tetrakis(triphenylphosphine)-palladium(0) (11 mg, 0.0096 mmol) in THF (0.8 ml) was added 0.4 ml of aqueous 1 N K2CO3 solution. The resultant mixture was heated at 160° C. under microwave irradiation for 10 min. The mixture was diluted with EtOAc (10 ml), and the organic phase was separated out. After removal of solvent, the residue was subjected to flash column chromatography on silica gel (elution with hexane-EtOAc) to yield LG2-75 as a white foam (35 mg). ESI-MS: m/z 419 (M+1); 1H NMR (600 MHz, CDCl3): δ 8.18 (1H, s), 8.04 (1H, d, J=7.8 Hz), 7.99 (1H, s), 7.78 (1H, d, J=7.8 Hz), 7.70 (1H, s), 7.69 (1H, d, J=4.8 Hz), 7.66 (1H, t, J=6.8 Hz), 7.52 (1H, d, J=4.8 Hz), 7.21 (2H, s), 3.92 (6H, s), 3.85 (3H, s), 2.69 (3H, s); 13C NMR (150.9 MHz, CDCl₃): δ 197.41, 153.43, 146.73, 138.11, 135.32, 133.93, 133.90, 132.36, 132.15, 132.08, 131.33, 129.74, 129.13, 129.07, 128.61, 128.56, 127.56, 108.95, 97.43, 61.05, 56.17, 26.78.

FIG. 9 illustrates a synthesis scheme for LG2-87 and LG2-89. To prepare LG2-87, a solution of 3,5-dibromo-imidazo[1,2-a]pyrazine (0.5 g, 1.84 mmol), 5-aminoindole (1.21 g, 9.2 mmol), and N,N-diisopropylethylamine (1.65 ml, 9.5 mmol) in CF3CH2OH (6.5 ml) was heated at 90° C. under microwave irradiation for 5 hours. The volatiles were evaporated, and the residue was subjected to flash column chromatography on silica gel (elution with CH₂Cl₂-MeOH) to yield LG2-87 as a white solid (35 mg, 6%). ESI-MS: m/z 328 (M+1), 330 (M+2+1); ¹H NMR (600 MHz, CDCl₃-MeOH-d4 85:15 v/v): δ 8.07 (1H, d, J=1.8 Hz), 7.56 (1H, s), 7.55 (1H, d, J=4.8 Hz), 7.51 (1H, d, J=4.8 Hz), 7.43 (1H, d, J=8.4 Hz), 7.38 (1H, dd, J=1.8, 9.0 Hz), 7.25 (1H, d, J=3.0 Hz), 6.52 (1H, d, J=3.0 Hz); ¹³C NMR (150.9 MHz, CDCl₃-MeOH-d4 85:15 v/v): δ 147.21, 134.05, 133.79, 131.96, 130.71, 129.62, 128.45, 125.73, 117.11, 113.39, 111.77, 108.66, 102.08, 98.64.

For preparation of LG2-89, to a solution of LG2-87 (25 mg, 0.076 mmol), 2-(1-naphthylene)-4,4,5,5-tetramethyl-1,3,2-dioxaborolane (39 mg, 0.152 mmol, 2.0 equiv.), and Tetrakis(triphenylphosphine)-palladium(0) (11 mg, 0.0096 mmol) in THF (1.0 ml) was added 0.5 ml of aqueous 1 N K2CO3 solution. The resultant mixture was heated at 160° C. under microwave irradiation for 10 min. The mixture was diluted with EtOAc (10 ml), and the organic phase was separated out. After removal of solvent, the residue was subjected to flash column chromatography on silica gel (elution with hexane-EtOAc) to yield LG2-89 as a white foam (33 mg). ESI-MS: m/z 376 (M+1); ¹H NMR (600 MHz, CDCl₃): δ 8.23 (1H, s), 8.18 (1H, br. s), 8.11 (1H, s), 8.02 (1H, br. s), 7.98 (1H, d, J=7.8 Hz), 7.73 (1H, s), 7.62 (3H, m), 7.57 (1H, t, J=7.2 Hz), 7.52 (1H, d, J=8.4 Hz), 7.49 (1H, t, J=7.8 Hz), 7.42 (1H, d, J=9.0 Hz), 7.39 (1H, d, J=4.2 Hz), 7.23 (1H, s), 7.13 (1H, d, J=4.2 Hz), 6.59 (1H, s); 13C NMR (150.9 MHz, CDCl₃): δ 147.36, 133.91, 133.84, 132.91, 132.22, 131.98, 131.78, 129.94, 129.10, 128.70, 128.63, 128.32, 127.13, 126.58, 126.51, 125.61, 125.49, 125.20, 124.93, 117.03, 112.68, 111.27, 109.27, 103.01.

FIG. 10 illustrates a synthesis scheme for LG2-60 and LG2-65. To prepare LG2-60, a solution of 3,5-dibromo-imidazo[1,2-a]pyrazine (1.0 g, 3.64 mmol), 4-isopropylaniline (2.46 g, 18.22 mmol), and N,N-diisopropylethylamine (3.3 ml, 18.94 mmol) in CF3CH2OH (12.5 ml) was heated at 90° C. under microwave irradiation for 5 hours. The volatiles were evaporated, and the residue was subjected to flash column chromatography on silica gel (elution with hexane-EtOAc) to yield LG2-60 as a brown gel (315 mg, 26%). ESI-MS: m/z 330 (M+1), 333 (M+2+1); 1H NMR (600 MHz, CDCl₃): δ 7.88 (1H, s), 7.72 (2H, d, J=8.4 Hz), 7.56 (1H, d, J=4.2 Hz), 7.54 (1H, s), 7.52 (1H, d, J=4.2 Hz), 7.25 (2H, d, J=8.4 Hz), 2.91 (1H, m), 1.26 (6H, d, J=6.6 Hz); ¹³C NMR (150.9 MHz, CDCl₃): δ 146.24, 144.12, 136.46, 133.82, 132.23, 129.22, 127.00, 120.05, 108.99, 98.15, 33.63, 24.09.

To synthesize LG2-65, to a solution of LG2-60 (83 mg, 0.25 mmol), 3-(4,4,5,5-tetramethyl-1,3,2-dioxaborolan-2-yl)-phenol (83 mg, 0.375 mmol, 1.5 equiv.), and Tetrakis(triphenylphosphine)-palladium(0) (35 mg, 0.03 mmol) in THF (2.5 ml) was added 1.2 ml of aqueous 1 N K2CO3 solution. The resultant mixture was heated at 160° C. under microwave irradiation for 10 min. The mixture was diluted with EtOAc (20 ml), and the organic phase was separated out. After removal of solvent, the residue was redissolved in dichloromethane, and the resultant white solids were collected to yield LG2-65 as a white solid (44 mg, 51%). ESI-MS: m/z 345 (M+1); 1H NMR (600 MHz, CDCl3-MeOH-d4 85:15 v/v): δ 7.85 (1H, d, J=4.8 Hz), 7.74 (2H, d, J=8.4 Hz), 7.65 (1H, s), 7.43 (1H, d, J=4.8 Hz), 7.38 (1H, t, J=7.8 Hz), 7.25 (2H, d, J=8.4 Hz), 7.08 (1H, d, J=7.2 Hz), 7.03 (1H, br. s), 6.91 (1H, dd, J=1.8, 8.4 Hz), 2.91 (1H, m), 1.27 (6H, d, J=7.2 Hz); 13C NMR (150.9 MHz, CDCl3-MeOH-d4 85:15 v/v): δ 157.87, 146.50, 143.97, 136.83, 133.20, 130.29, 130.00, 129.12, 128.92, 128.20, 126.60, 120.28, 119.05, 115.82, 114.70, 109.24, 33.55, 23.50.

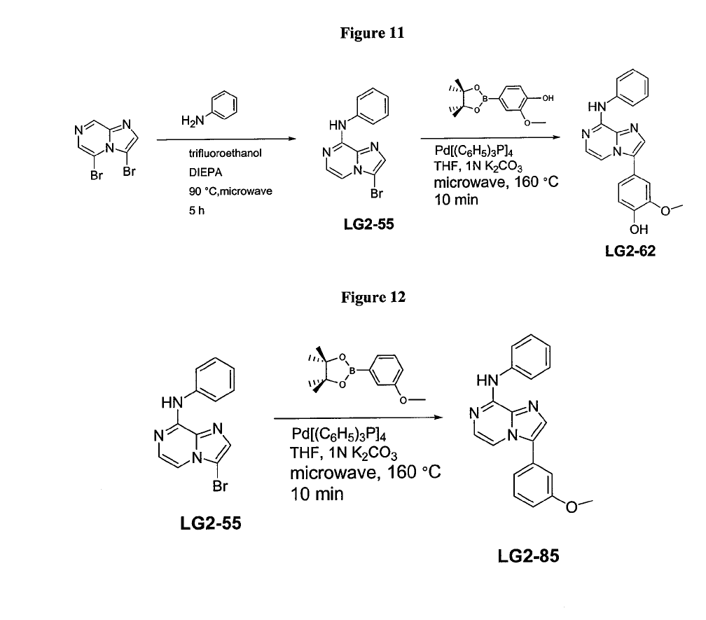

FIG. 11 illustrates a synthesis scheme for LG2-55 and LG2-62. To prepare LG2-55, a solution of 3,5-dibromo-imidazo[1,2-a]pyrazine (1.0 g, 3.64 mmol), aniline (1.70 g, 18.22 mmol), and N,N-diisopropylethylamine (3.3 ml, 18.94 mmol) in CF3CH2OH (12.5 ml) was heated at 90° C. under microwave irradiation for 5 hours. The volatiles were evaporated, and the residue was subjected to flash column chromatography on silica gel (elution with hexane-EtOAc) to yield LG2-55 as a white crystalline solid (109 mg, 10%). ESI-MS: m/z 289 (M+1), 291 (M+2+1); ¹H NMR (600 MHz, CDCl₃): δ 7.94 (1H, s, NH), 7.84 (2H, d, J=8.4 Hz), 7.58 (1H, d, J=4.2 Hz), 7.55 (1H, s), 7.54 (1H, d, J=4.2 Hz), 7.39 (2H, t, J=7.8 Hz), 7.10 (1H, t, J=7.8 Hz); 13C NMR (150.9 MHz, CDCl₃): δ 146.09, 138.90, 133.80, 132.31, 129.08 (3×C), 123.27, 119.65 (2×C), 109.25, 98.21.

To synthesize LG2-62, to a solution of LG2-55 (31 mg, 0.107 mmol), 2-methoxy-4-(4,4,5,5-tetramethyl-1,3,2-dioxaborolan-2-yl)-phenol (40 mg, 0.161 mmol, 1.5 equiv.), and Tetrakis(triphenylphosphine)-palladium(0) (15 mg, 0.013 mmol) in THF (1.0 ml) was added 0.5 ml of aqueous 1 N K2CO3 solution. The resultant mixture was heated at 160° C. under microwave irradiation for 10 min. The mixture was diluted with EtOAc (15 ml), and the organic phase was separated out. After removal of solvent, the residue was subjected to flash column chromatography on silica gel (elution with CH₂Cl₂-MeOH) to yield LG2-62 as a white solid (19 mg, 54%). ESI-MS: m/z 333 (M+1); ¹H NMR (600 MHz, CDCl₃): δ 8.03 (1H, s, NH), 7.87 (2H, d, J=7.8 Hz), 7.67 (1H, d, J=4.8 Hz), 7.57 (1H, s), 7.48 (1H, d, J=4.8 Hz), 7.39 (2H, t, J=7.8 Hz), 7.09 (1H, t, J=7.8 Hz), 7.08 (2H, s), 7.01 (1H, s), 5.83 (1H, s, OH), 3.97 (3H, s, OCH3); ¹³C NMR (150.9 MHz, CDCl₃): δ 148.98, 147.12, 146.64, 146.38, 139.32, 130.45, 129.07, 128.46, 122.98, 121.69, 120.33, 119.56, 115.20, 110.92, 109.31, 56.18.

FIG. 12 illustrates a synthesis scheme for LG2-85. To a solution of LG2-55 (32 mg, 0.110 mmol), 2-(3-Methoxyphenyl)-4,4,5,5-tetramethyl-1,3,2-dioxaborolane (39 mg, 0.166 mmol, 1.5 equiv.), and Tetrakis(triphenylphosphine)-palladium(0) (11 mg, 0.010 mmol) in THF (1.0 ml) was added 0.5 ml of aqueous 1 N K2CO3 solution. The resultant mixture was heated at 160° C. under microwave irradiation for 10 min. The mixture was diluted with EtOAc, and the organic phase was separated out. After removal of solvent, the residue was subjected to flash column chromatography on silica gel (elution with hexane-EtOAc) to yield LG2-85 as a white foam (32 mg, 91%). ESI-MS: m/z 317 (M+1); 1H NMR (600 MHz, CDCl₃): δ 8.06 (1H, s, NH), 7.87 (2H, d, J=7.8 Hz), 7.74 (1H, d, J=4.8 Hz), 7.64 (1H, s), 7.49 (1H, d, J=4.8 Hz), 7.45 (1H, t, J=7.8 Hz), 7.39 (2H, t, J=7.8 Hz), 7.16 (1H, d, J=7.8 Hz), 7.09 (2H, m), 7.00 (1H, dd, J=7.8, 3.0 Hz), 3.87 (3H, s); 13C NMR (150.9 MHz, CDCl₃): δ 160.24, 146.69, 139.27, 133.80, 130.95, 130.42, 129.70, 129.06, 128.59, 123.00, 120.31, 119.59, 114.10, 113.83, 109.44, 55.45.

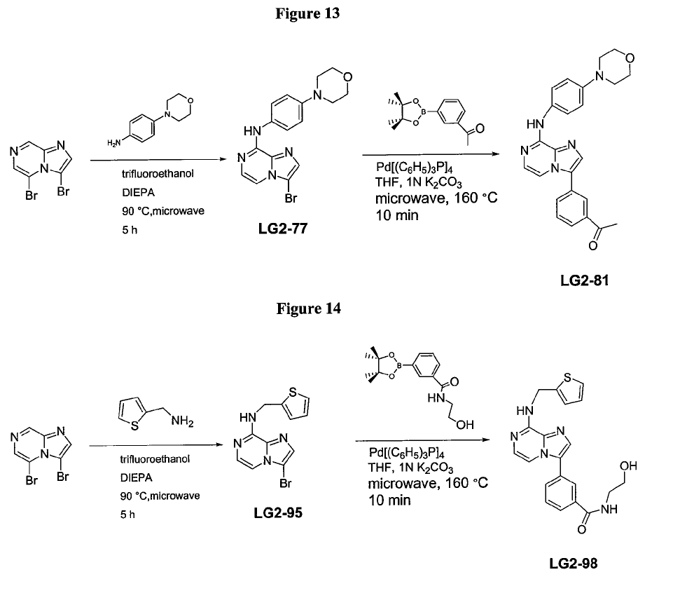

FIG. 13 illustrates a synthesis scheme for LG2-77 and LG2-81. To prepare LG2-77, a solution of 3,5-dibromo-imidazo[1,2-a]pyrazine (0.52 g, 1.84 mmol), 4-morpholinoaniline (1.64 g, 9.1 mmol), and N,N-diisopropylethylamine (1.65 ml) in CF3CH2OH (6.5 ml) was heated at 90° C. under microwave irradiation for 5 hours. The volatiles were evaporated, and the residue was subjected to flash column chromatography on silica gel (elution with hexane-EtOAc) to yield LG2-77 as a brown crystalline solid (97 mg, 14%). ESI-MS: m/z 374 (M+1), 376 (M+2+1); 1H NMR (600 MHz, CDCl₃): δ 7.81 (1H, s, NH), 7.69 (2H, d, J=9.0 Hz), 7.54 (2H, d+s), 7.50 (1H, d, J=6.4 Hz), 6.95 (2H, d, J=9.0 Hz), 3.87 (4H, t, J=6.4 Hz), 3.14 (4H, t, J=6.4 Hz); ¹³C NMR (150.9 MHz, CDCl₃): δ 147.75, 146.34, 133.81, 132.17, 131.59, 129.28, 121.48, 116.63, 108.75, 98.11, 66.96, 49.96. To synthesize LG2-81, to a solution of LG2-77 (34 mg, 0.091 mmol), 143-(4,4,5,5-tetramethyl-1,3,2-dioxaborolan-2-yl)phenyl]-ethanone (33 mg, 0.136 mmol, 1.5 equiv.) and Tetrakis(triphenylphosphine)-palladium(0) (10 mg, 0.013 mmol) in THF (1.0 ml) was added 0.5 ml of aqueous 1 N K2CO3 solution. The resultant mixture was heated at 160° C. under microwave irradiation for 10 min. The mixture was diluted with EtOAc, and the organic phase was separated out. After removal of solvent, the residue was subjected to flash column chromatography on silica gel (elution with CH2Cl2-MeOH) to yield LG2-81 as a pale yellow solid (18 mg, 48%). ESI-MS: m/z 414 (M+1); ¹H NMR (600 MHz, CDCl₃): δ 8.16 (1H, s, NH), 8.02 (1H, d, J=7.8 Hz), 7.97 (1H, s), 7.77 (1H, d, J=7.8 Hz), 7.73 (2H, d, J=9.0 Hz), 7.67 (1H, s), 7.65 (2H, d+t), 7.48 (1H, d, J=6.4 Hz), 6.96 (2H, d, J=9.0 Hz), 3.87 (4H, t, J=6.4 Hz), 3.14 (4H, t, J=6.4 Hz), 2.69 (3H, s); ¹³C NMR (150.9 MHz, CDCl₃): δ 197.41, 147.63, 146.97, 138.08, 134.00, 132.33, 131.91, 131.22, 129.68, 129.24, 129.21, 128.48, 127.57, 121.43, 116.66, 108.52, 66.97, 50.00, 26.77.

FIG. 14 shows a synthesis scheme for LG2-95 and LG2-98. To prepare LG2-95, a solution of 3,5-dibromo-imidazo[1,2-a]pyrazine (0.52 g, 1.84 mmol), thiophene-2-methylamine (1.04 g, 9.2 mmol), and N,N-diisopropylethylamine (1.65 ml) in CF3CH2OH (6.5 ml) was heated at 90° C. under microwave irradiation for 5 hours. The volatiles were evaporated, and the residue was subjected to flash column chromatography on silica gel (elution with hexane-EtOAc) to yield LG2-95 as a pale yellow solid (110 mg, 19%). ESI-MS: m/z 309 (M+1), 311 (M+2+1); ¹H NMR (600 MHz, CDCl₃): δ 7.51 (1H, d, J=4.8 Hz), 7.46 (1H, s), 7.44 (1H, d, J=4.8 Hz), 7.22 (1H, d, J=5.4 Hz), 7.07 (1H, d, J=3.0 Hz), 6.96 (1H, dd, J=5.4, 3.0 Hz), 4.96 (211, d, J=6.0 Hz).

To synthesize LG2-98, to a solution of LG2-95 (34 mg, 0.110 mmol), N-(2-hydroxyethyl)-3-(4,4,5,5-tetramethyl-1,3,2-dioxaborolan-2-yl)-benzamide (63 mg, 0.203 mmol, 1.85 equiv.) and Tetrakis(triphenylphosphine)-palladium(0) (147 mg, 0.127 mmol, 1.15 equiv) in THF (1.0 ml) was added 0.5 ml of aqueous 1 N K2CO3 solution. The resultant mixture was heated at 160° C. under microwave irradiation for 10 min. The mixture was diluted with EtOAc, and the organic phase was separated out. After removal of solvent, the residue was subjected to flash column chromatography on silica gel (elution with CH₂Cl₂-acetone) to yield LG2-98 as a white solid (8.8 mg, 20%). ESI-MS: m/z 394 (M+1); ¹H NMR (600 MHz, CDCl₃): δ 8.00 (1H, br. s), 7.81 (1H, d, J=7.8 Hz), 7.69 (1H, d, J=7.8 Hz), 7.60 (3H, m), 7.44 (1H, d, J=4.8 Hz), 7.23 (1H, dd, J=0.9, 5.1 Hz), 7.10 (1H, d, J=3.0 Hz), 6.97 (1H, dd, J=5.1, 3.0 Hz), 5.00 (2H, d, J=5.4 Hz), 3.88 (2H, dd, J=4.8, 9.6 Hz), 3.68 (2H, J=4.8, 9.6 Hz); 13C NMR (150.9 MHz, CDCl₃): δ 167.58, 148.88, 141.20, 135.46, 133.75, 131.05, 130.80, 129.51, 129.25, 129.22, 127.34, 126.85, 126.74, 126.72, 126.10, 125.09, 107.94, 62.09, 42.85, 39.49.

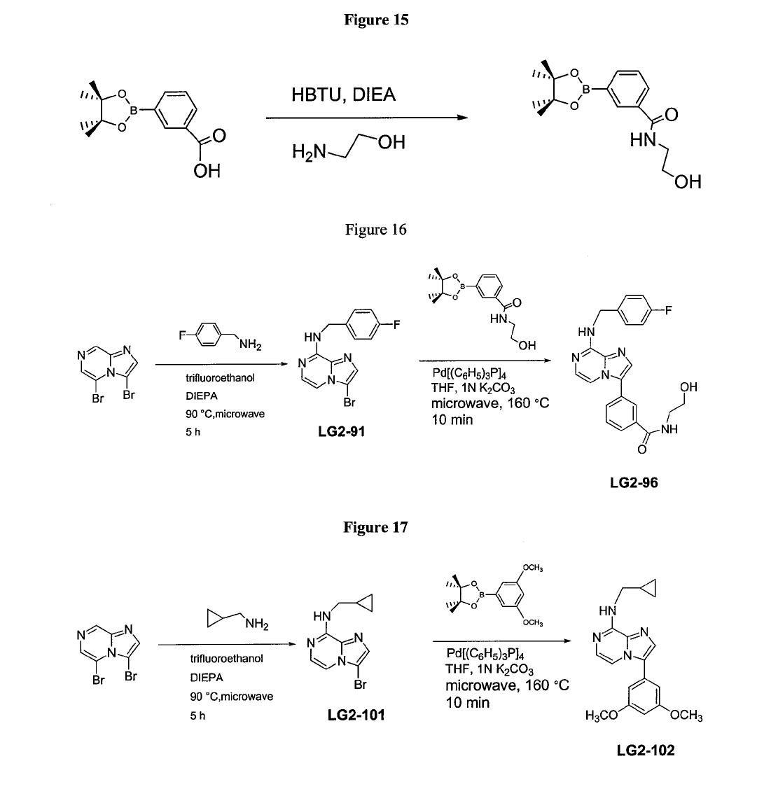

FIG. 15 shows a synthesis scheme for N-(2-hydroxyethyl)-3-(4,4,5,5-tetramethyl-1,3,2-dioxaborolan-2-yl)-benzamide. To a solution of 3-(4,4,5,5-tetramethyl-1,3,2-dioxaborolan-2-yl)-benzoic acid (1.15 g, 4.63 mmol) and HBTU (1.52 g, 4.03 mmol) in anhydrous DMF (20 ml) was added N,N-diisopropylethylamine (1.2 ml, 6.71 mmol). The resultant solution was stirred at room temperature for 45 min. Ethanolamine (0.29 ml, 4.84 mmol) was added slowly to the solution, and the stirring was continued for another 25 h. After evaporation of solvent, the residue was subjected to flash column chromatography on silica gel (elution with CH₂Cl₂-EtOAC) to yield N-(2-hydroxyethyl)-3-(4,4,5,5-tetramethyl-1,3,2-dioxaborolan-2-yl)-benzamide as white crystals (398 mg). ESI-MS: m/z 292 (M+1); in NMR (600 MHz, CDCl₃): δ 8.11 (1H, s), 7.98 (1H, d, J=7.2 Hz), 7.94 (1H, d, J=7.2 Hz), 7.46 (1H, t, J=7.2 Hz), 3.85 (2H, t, J=4.8 Hz), 3.64 (2H, dd, J=4.8, 10.2 Hz); ¹³C NMR (150.9 MHz, CDCl₃): δ 168.69, 138.00, 133.42, 132.39, 130.52, 128.21, 84.19, 62.47, 42.97, 24.88.

FIG. 16 shows synthesis of LG2-91 and LG2-96. To prepare LG2-91, a solution of 3,5-dibromo-imidazo[1,2-a]pyrazine (0.52 g, 1.84 mmol), 4-fluorobenzylamine (1.15 g, 9.2 mmol), and N,N-diisopropylethylamine (1.65 ml) in CF3CH2OH (6.5 ml) was heated at 90° C. under microwave irradiation for 5 hours. The volatiles were evaporated, and the residue was subjected to flash column chromatography on silica gel (elution with hexane-EtOAc) to yield LG2-91 as a pale yellow solid (143 mg, 24%). ESI-MS: m/z 321 (M+1), 323 (M+2+1); 1H NMR (600 MHz, CDCl₃): δ 7.48 (1H, d, J=4.8 Hz), 7.46 (1H, s), 7.44 (1H, d, J=4.8 Hz), 7.36 (2H, dd, J=8.4, 5.4 Hz), 7.02 (2H, t, J=8.4 Hz), 4.76 (2H, d, J=5.4 Hz).

To synthesize LG2-96, to a solution of LG2-91 (39 mg, 0.121 mmol), N-(2-hydroxyethyl)-3-(4,4,5,5-tetramethyl-1,3,2-dioxaborolan-2-yl)-benzamide (99 mg, 0.340 mmol, 2.8 equiv.) and Tetrakis(triphenylphosphine)-palladium(0) (49 mg, 0.042 mmol) in THF (1.0 ml) was added 0.5 ml of aqueous 1 N K2CO3 solution. The resultant mixture was heated at 160° C. under microwave irradiation for 10 min. The mixture was diluted with EtOAc, and the organic phase was separated out. After removal of solvent, the residue was subjected to flash column chromatography on silica gel (elution with CH₂Cl₂-MeOH) to yield LG2-96 as a pale brown foam (37 mg, 73%). ESI-MS: m/z 406 (M+1); 1H NMR (600 MHz, CDCl₃): δ 8.01 (1H, s), 7.80 (1H, d, J=7.8 Hz), 7.68 (1H, d, J=7.2 Hz), 7.60 (3H, m), 7.40 (3H, m), 7.03 (2H, t, J=8.4 Hz), 6.67 (111, br. s, OH), 6.34 (1H, t, J=5.4 Hz, NH), 4.79 (2H, d, J=5.4 Hz), 3.87 (2H, t, J=5.4 Hz), 3.68 (2H, J=5.4, 10.2 Hz); ¹³C NMR (150.9 MHz, CDCl₃): δ 167.61, 162.17 (d, 1JC-F=245 Hz), 149.14, 135.41, 134.29, 133.66, 130.82, 130.67, 129.47, 129.41, 129.30, 128.99, 127.38, 126.86, 126.69, 115.45 (d, 2JC-F=21 Hz), 107.76, 61.78, 43.97, 42.87.

FIG. 17 shows a synthesis scheme for LG2-101 and LG2-102. To prepare LG2-101, a solution of 3,5-dibromo-imidazo[1,2-a]pyrazine (0.52 g, 1.84 mmol), aminomethylpropane (0.65 g, 9 2 mmol), and N,N-diisopropylethylamine (1.65 ml) in CF3CH2OH (6.5 ml) was heated at 90° C. under microwave irradiation for 5 hours. The volatiles were evaporated, and the residue was subjected to flash column chromatography on silica gel (elution with hexane-EtOAc) to yield LG2-101 as a pale yellow solid (120 mg, 24%). ESI-MS: m/z 267 (M+1), 269 (M+2+1); ¹H NMR (600 MHz, CDCl₃): δ 7.47 (1H, s), 7.44 (1H, d, J=4.8 Hz), 7.39 (1H, d, J=4.8 Hz), 3.43 (2H, dd, J=5.4, 6.6 Hz), 1.16 (1H, m), 0.58 (2H, m), 0.31 (2H, m); 13C NMR (150.9 MHz, CDCl₃): δ 148.95, 133.74, 131.83, 129.58, 107.43, 97.76, 45.84, 10.62, 3.59.

To prepare LG2-102, to a solution of LG2-101 (32 mg, 0.119 mmol), 2-(3,5-dimethoxyphenyl)-(4,4,5,5-tetramethyl-1,3,2-dioxaborolane (47 mg, 0.178 mmol, 1.5 equiv.) and Tetrakis(triphenylphosphine)-palladium(0) (16 mg, 0.013 mmol) in THF (1.0 ml) was added 0.5 ml of aqueous 1 N K2CO3 solution. The resultant mixture was heated at 160° C. under microwave irradiation for 10 min. The mixture was diluted with EtOAc, and the organic phase was separated out. After removal of solvent, the residue was subjected to flash column chromatography on silica gel (elution with hexane-EtOAc) to yield LG2-102 as a white gel (16 mg, 41%). ESI-MS: m/z 325 (M+1); ¹H NMR (600 MHz, CDCl₃): δ 7.60 (1H, d, J=4.8 Hz), 7.55 (1H, s), 7.36 (1H, d, J=4.8 Hz), 6.673 (1H, s), 6.670 (1H, s), 6.53 (1H, s), 6.14 (1H, t, J=5.4 Hz, NH), 3.85 (6H, s), 3.46 (2H, dd, J=5.4, 12.0 Hz), 1.18 (1H, m), 0.59 (2H, m), 0.32 (2H, m); 13C NMR (150.9 MHz, CDCl₃): δ 161.41, 149.54, 133.76, 130.59, 130.47, 129.07, 128.25, 107.78, 106.16, 100.37, 55.54, 45.84, 10.71, 3.62.

LG-2 Compounds and HIF-1GFP/DsRFP expression and WST-1 test in C6#4 cells.

In this experiment were used Ephrin compounds: LG2-7; LG2-11; LG2-13; LG2-62; LG2-65; LG2-75; LG2-81; LG2-85; LG2-89; LG2-96:LG2-98; LG2-102. Px-478 HIF-1 inhibitor was used as positive control, DMSO—as negative control.•C6#4 cell line (after additional sorting) was growing into flasks with D-MEM/F-12 medium with 10% FBS and antibiotics at 37° C. in humidified atmosphere with 5% CO2. Cells were kept in the Log phase proliferate activity. On the day of experiment, C6#4 cells were transfer into in 96 well Costar plates 20×10̂3 cell/well in 100 μl/well media and cultivated for 24 hours, than cultural media was replaced with 100 μl/well media, containing the drugs in different concentrations with logarithmic dilutions from 200 μM to 0.1 μM and cultivated for additional 24 hours. TECAN (SAFIRE plate reader) was used for determination of the level of expression GFP and RFP. Excitation wavelength: 484 nm; Emission wavelength: 510 nm. Cell viability (proliferation) was estimated by and WST-1 4 h testing. 11 μl/well of Cell Proliferation Reagent (WST-1) was added into wells with cells, growing in 100 ul media and plates were incubated at 37° C. for additional 4 hours. Measure the absorbance was made by SAFIRE plate reader with wavelength 440 nm for measuring the absorbance of the formazan product, and wavelength 600 nm for the reference.

To analyze possible influence drugs on HIF-1 expression, we first normalized level of the GFP by subdividing data of the GFP expression on data of RFP expression (G/R data) and then compared it to cell viability detected by WST-1 test. We classified the compound as possibly involved in HIF-1 pathways if drug concentration down-regulated GFP expression, stimulated by CoCl₂, but did not influence on cell viability.

Statistical Analysis, Detection of the EC-50, and Graphics Made with Graph Pad Prism-4.

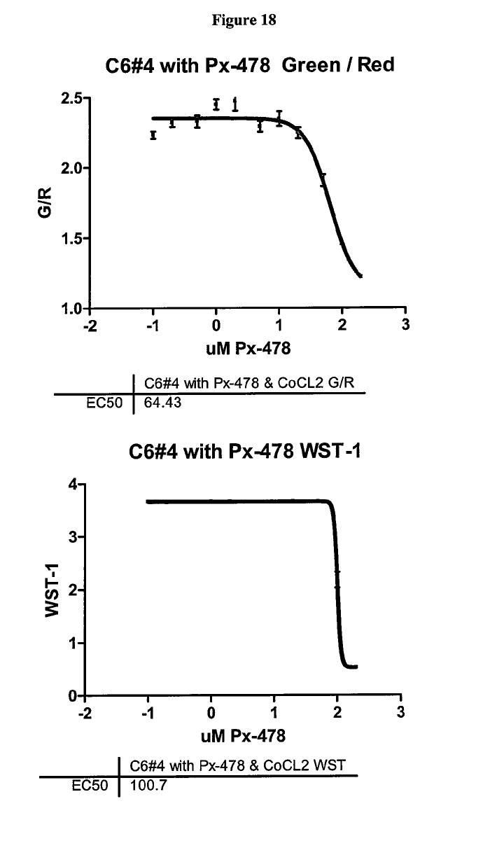

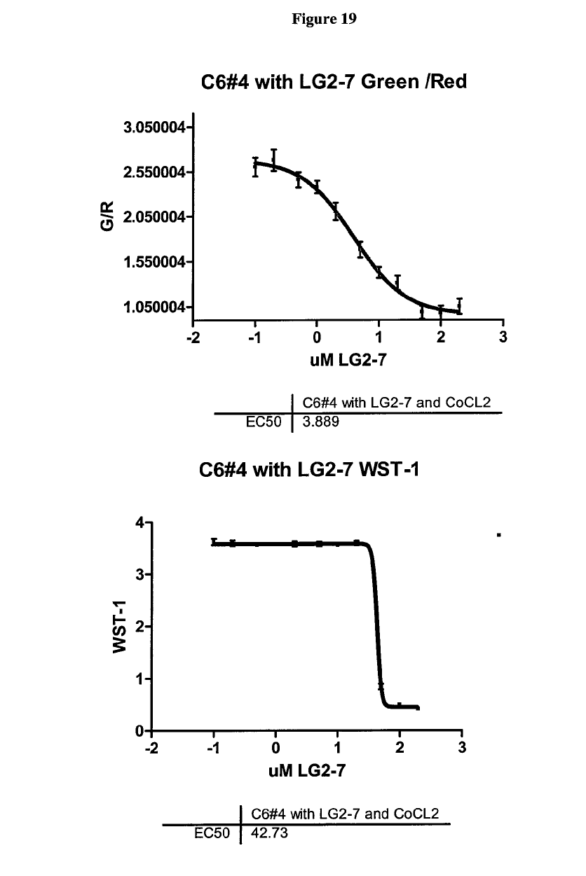

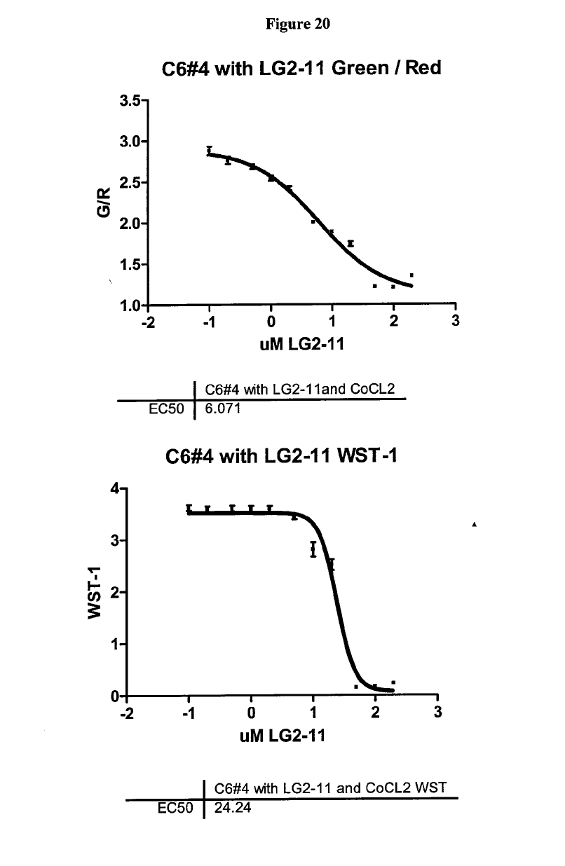

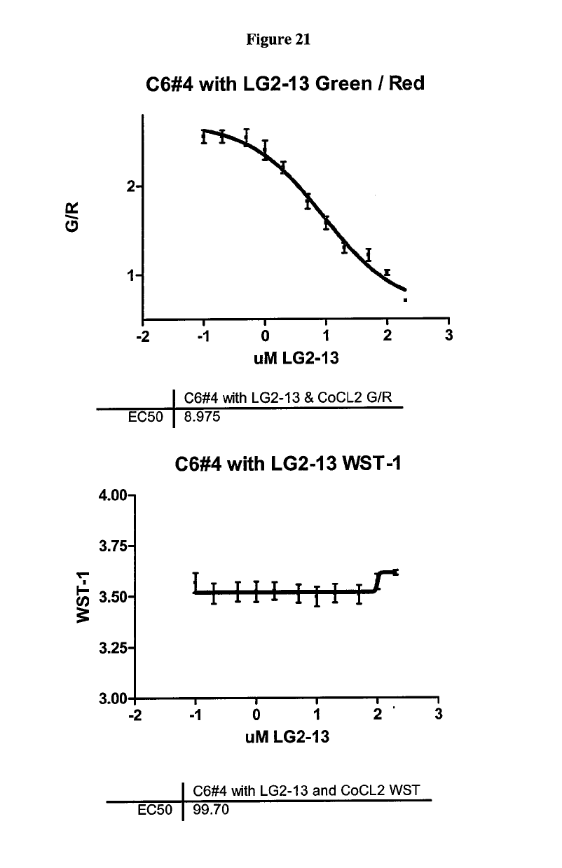

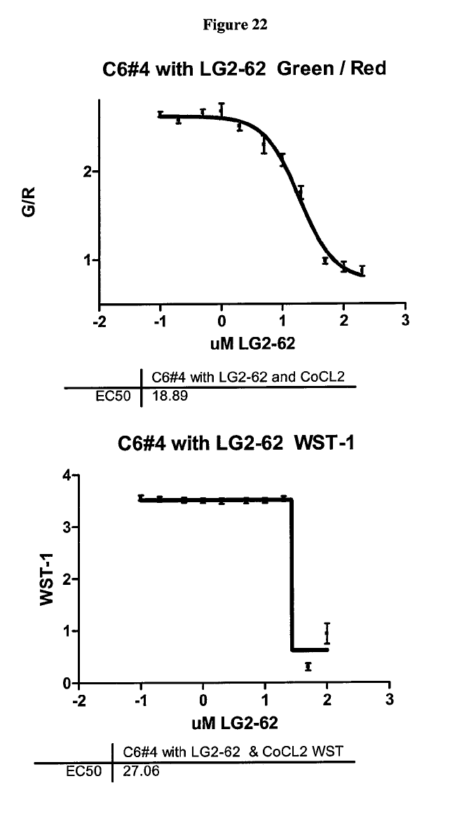

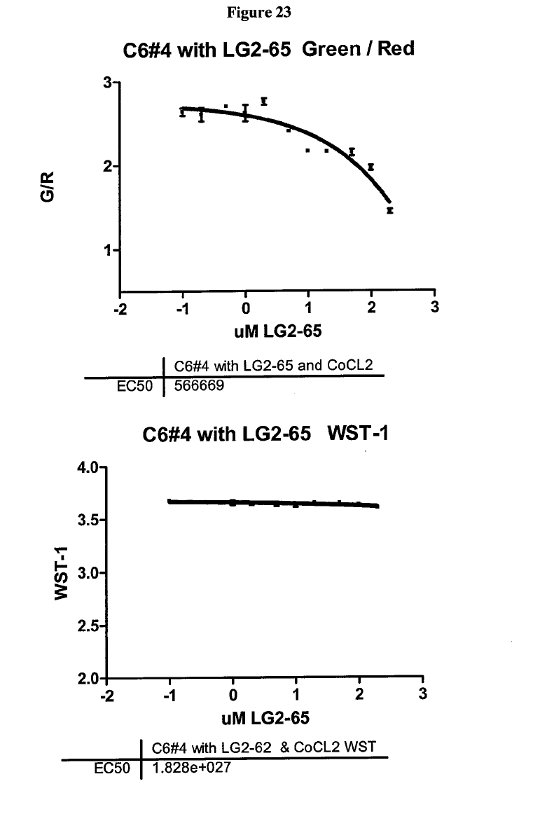

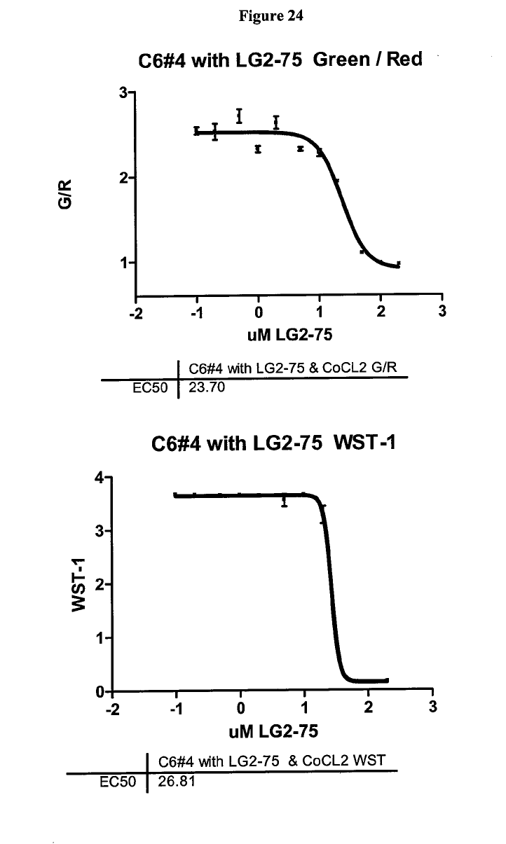

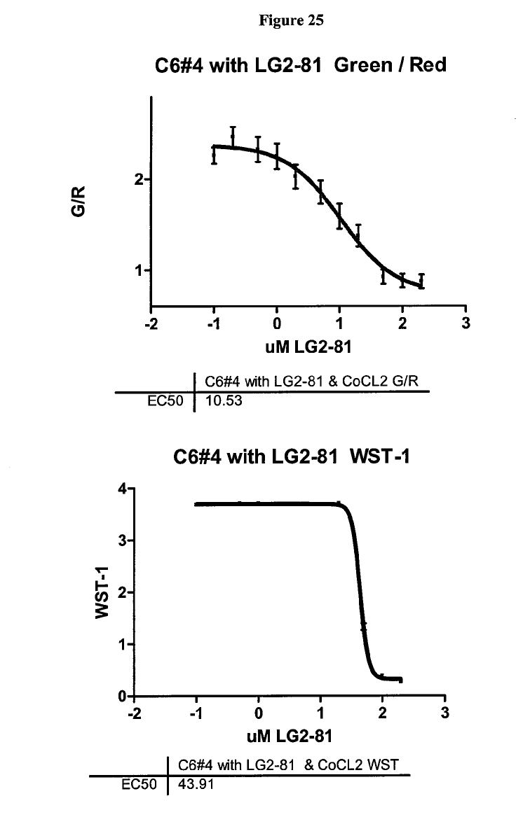

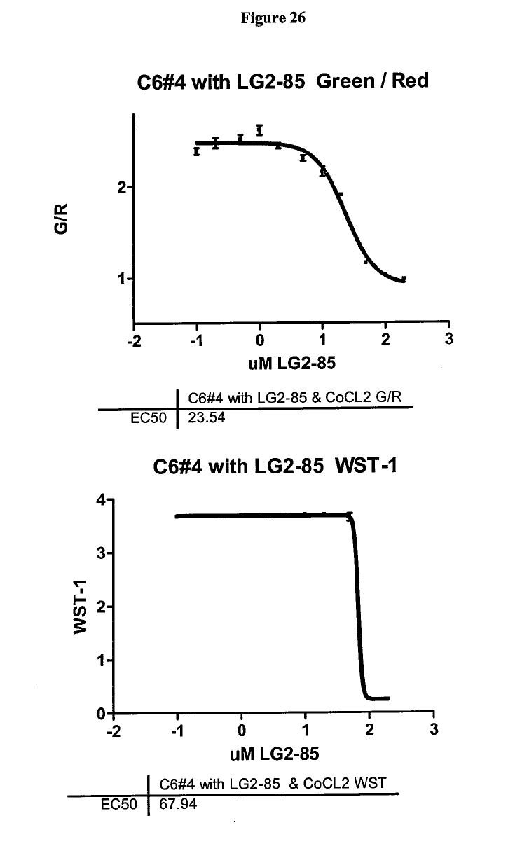

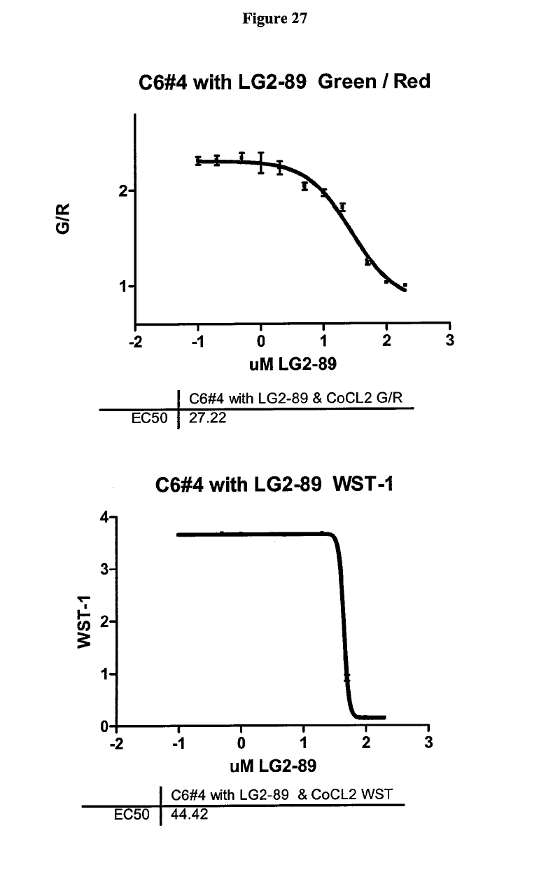

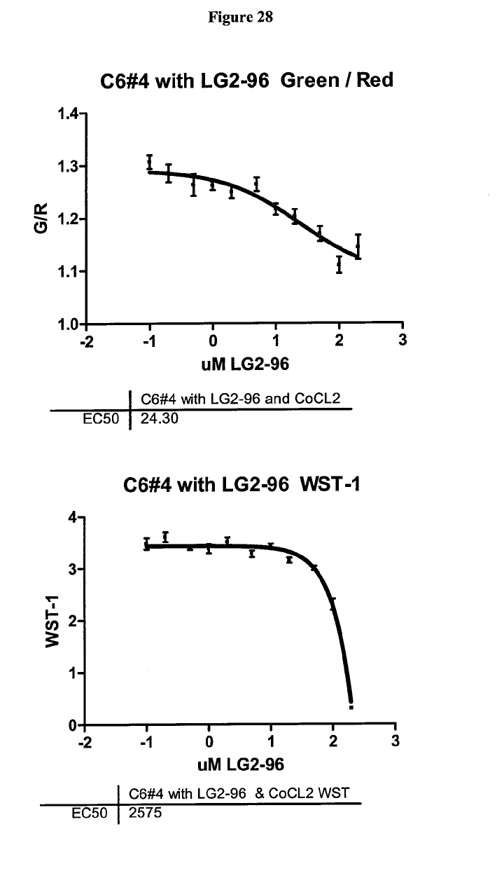

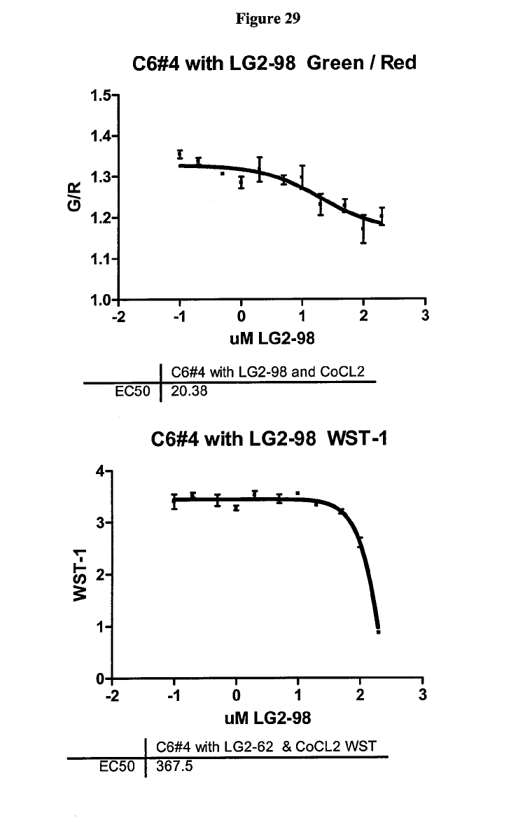

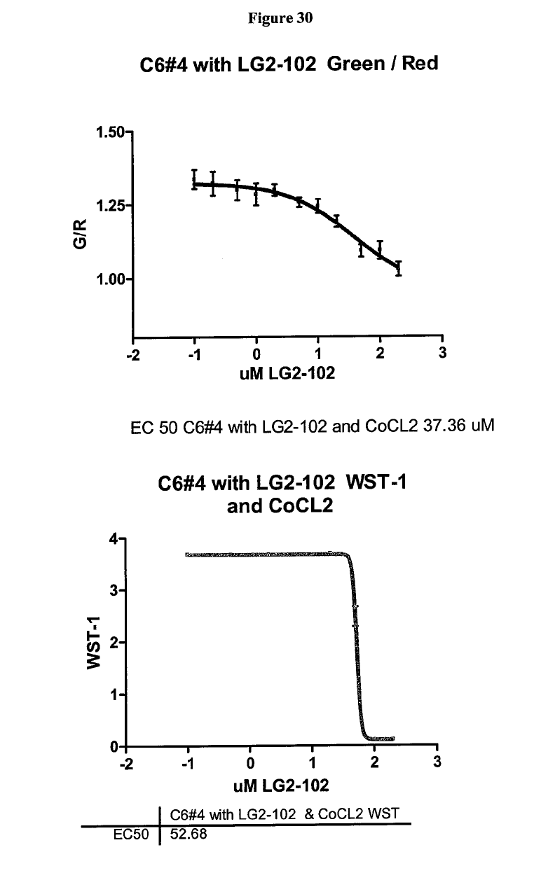

FIG. 18-FIG. 30 show the results of testing for HIF-1 expression (top) and viability (bottom). Table 2 below shows that the influence of LG2-compounds on HIF-1 is dependent upon GFP expression. LG2-13 doesn't have cytotoxicity, but may be involved in HIF-1. EC 50 G/R=7.95. LG2-65 doesn't influence on C6#4 cells at concentration 200 μM. Table 3 shows a summary of the effects of the different compound on C6#4 cells EC-50. Table 4 shows a summary of the average effects of LG2-compounds in terms of GFP/RFP expression and WST-testing.

TABLE 2
Influence of LG2-compounds on HIF-1

| | G/R (EC-50) | WST-1 (EC-50) | G/R:WST-1 |
|---|---|---|---|
| Compounds | μM | μM | Ratio |
| Px-478 | 45.2 | 88.2 | 2.1 |
| LG2-7 | 9.1 | 35.2 | 5.6 |
| LG2-11 | 2.2 | 19.6 | 2.2 |
| LG2-13 | 7.95 | — | — |
| LG2-62 | 18.9 | 30.1 | 1.5 |
| LG2-75 | 14.77 | 16.76 | 1.2 |
| LG2-81 | 10.5 | 38.8 | 3.7 |
| LG2-85 | 23.5 | 48.1 | 2.0 |
| LG2-89 | 27.9 | 35.4 | 1.4 |
| LG2-96 | 24.3 | 2575? | ? |
| LG2-98 | 20.38 | 367? | ? |
| LG2-102 | 37.36 | 52.68 | 1.4 |

TABLE 3
Summary of Effects of Different Inhibitor Compounds on C6#4 Cells

| | G/R | G/R | G/R | WST-1 | WST-1 | WST-1 | G/R:WST-1 | G/R:WST-1 | G/R:WST-1 | G/R:WST-1 |
|---|---|---|---|---|---|---|---|---|---|---|
| Com- | (EC-50) | (EC-50) | (EC-50) | (EC-50) | (EC-50) | (EC-50) | Ratio | Ratio | Ratio | Ratio |
| pound | 1 ex | 2 ex | 3 ex | 1 ex | 2 ex | 3 ex | 1 ex | 2 ex | 3 ex | Mean |
| Px-478 | 27.87 | 43.30 | 64.43 | 80.24 | 83.54 | 100.7 | 2.9 | 1.9 | 1.6 | 2.1 |
| LG2-7 | 14.22 | 9.34 | 3.889 | 23 | 39.89 | 42.73 | 1.6 | 4.3 | 11 | 5.6 |
| LG2-11 | 10.88 | 15.74 | 6.07 | 18.05 | 16.56 | 24.24 | 1.66 | 1.05 | 4 | 2.2 |
| LG2-13 | 2.61e+006 | 6.9 | 8.975 | 7.084 | 101 | 99.7 | — | — | — | — |
| LG2-62 | 13.45 | 24.45 | 18.89 | 19.47 | 43.86 | 27.06 | 1.4 | 1.8 | 1.4 | 1.5 |
| LG2-65 | 9072 | 3857 | 5666.69 | 56.39 | 2.567e+012 | 1.828e+027 | — | — | — | — |
| LG2-75 | 4.365 | 16.27 | 23.7 | 7.044 | 16.43 | 26.81 | 1.6 | 1 | 1.1 | 1.2 |
| LG2-81 | 8.659 | 12.22 | 10.53 | 32.21 | 40.18 | 43.91 | 3.7 | 3.2 | 4.2 | 3.7 |
| LG2-85 | 23.89 | 23.05 | 23.54 | 34.81 | 41.56 | 67.94 | 1.4 | 1.8 | 2.9 | 2.0 |
| LG2-89 | 13.72 | 42.70 | 27.22 | 21.40 | 40.43 | 44.42 | 1.6 | 0.95 | 1.6 | 1.4 |

TABLE 4
Summary of Average Effects of LG2-compounds on C6#4 Cells
(Mean and Standard Deviation)

| | G/R | G/R | WST-1 | WST-1 | G/R:WST-1 | G/R:WST-1 |
|---|---|---|---|---|---|---|
| | (REC-50) | (REC-50) | (EC-50) | (EC-50) | Ratio | Ratio |
| Compound | Mean | SD | Mean | SD | Mean | SD |
| Px-478 | 45.2 | 18.35391 | 88.160 | 10.98459 | 2.133333 | 0.6806859 |
| LG2-7 | 9.149667 | 5.168129 | 35.20667 | 10.66623 | 5.633333 | 4.839766 |
| LG2-11 | 2.236667 | 1.557252 | 19.61667 | 4.072654 | 2.236667 | 1.557242 |
| LG2-13 | | | | | | |
| LG2-62 | 18.930 | 5.500109 | 30.130 | 12.48145 | 1.533333 | 0.2309401 |
| LG2-65 | | | | | | |
| LG2-75 | 14.77833 | 9.753428 | 16.76133 | 9.887164 | 1.233333 | 0.321455 |
| LG2-81 | 10.46967 | 1.781266 | 38.7667 | 5.976675 | 3.700 | 0.4999999 |
| LG2-85 | 23.49333 | 0.4219401 | 48.13334 | 17.4907 | 2.033333 | 0.7767454 |
| LG2-89 | 27.880 | 14.50127 | 35.41667 | 12.30164 | 1.383333 | 0.3752777 |

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

### REFERENCES

Kaplan, W., Littlejohn, T. G. (2001) Swiss-PDB Viewer (Deep View) Brief Bioinform 2, 195-7.
Guex, N., Peitsch, M. C. (1997) SWISS-MODEL and the Swiss-PdbViewer: an environment for comparative protein modeling Electrophoresis 18, 2714-23.
Kramer, B., Rarey, M., Lengauer, T. (1999) Evaluation of the FLEXX incremental construction algorithm for protein-ligand docking Proteins 37, 228-41.
Sybyl 7.1, Tripos, Inc., 1699 South Hanley Road, St. Louis, Mo., 63144, USA
Hindle, S. A., Rarey, M., Buning, C., Lengaue, T. (2002) Flexible docking under pharmacophore type constraints J Comput Aided Mol Des 16, 129-49.
Boehm, H.-J., Stahl, M. (2002) The use of scoring functions in drug discovery applications Reviews in Computational Chemistry 18, 41-87.
Fisher, M. J. A., Ann E.; Giese, Ulrich; Gunn, Bruce P.; Harms, Cathy S.; Khau, Vien; Kinnick, Michael D.; Lindstrom, Terry D.; Martinelli, Michael J.; Mest, Hans-Juergen; Mohr, Michael; Morin, John M., Jr.; Mullaney, Jeffrey T.; Nunes, Anne; Paal, Michael; Rapp, Achim; Ruehter, Gerd; Ruterbories, Ken J.; Sall, Daniel J.; Scarborough, Robert M.; Schotten, Theo; Sommer, Birgit; Stenzel, Wolfgang; Towner, Richard D.; Urn, Suzane L.; Utterback, Barbara G.; Vasileff, Robert T.; Voeelkers, Silke; Wyss, Va. L.; Jakubowski, Joseph A. Journal of Medicinal Chemistry 1999, 42, 4875-4889.

What is claimed is:

1. A composition for inhibiting catalytic activity of a tyrosine kinase comprising a compound represented by Formula (I) or an enantiomer, diastereomer, pharmaceutically acceptable salt, hydrate, prodrug, or solvate thereof 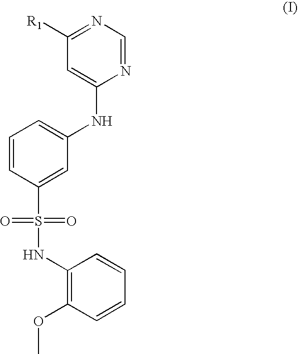
wherein R₁ is selected from the group consisting of the following structures:

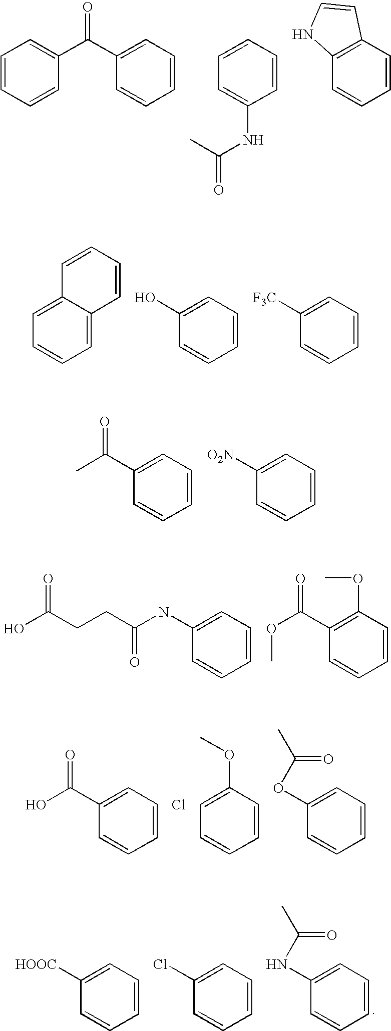

2. A composition for inhibiting catalytic activity of a tyrosine kinase comprising a compound represented by Formula (II) or an enantiomer, diastereomer, pharmaceutically acceptable salt, hydrate, prodrug, or solvate thereof 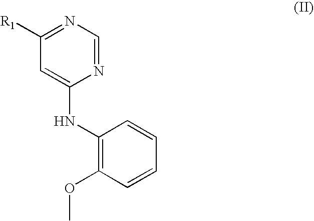
wherein R₁ is selected from the group consisting of the following structures

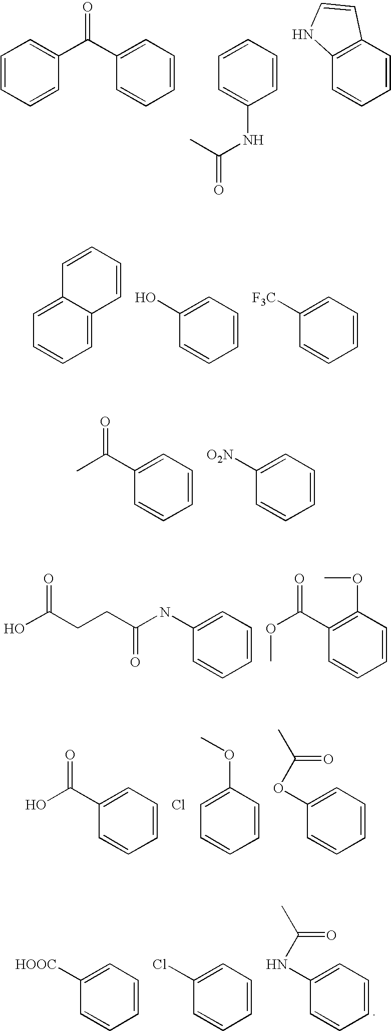

3. A composition for inhibiting catalytic activity of a tyrosine kinase comprising a compound of Formula (III) or an enantiomer, diastereomer, pharmaceutically acceptable salt, hydrate, prodrug, or solvate thereof 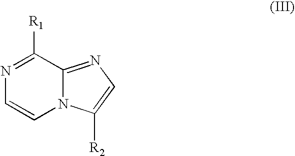
wherein R₁ is selected from the group consisting of the following structures: 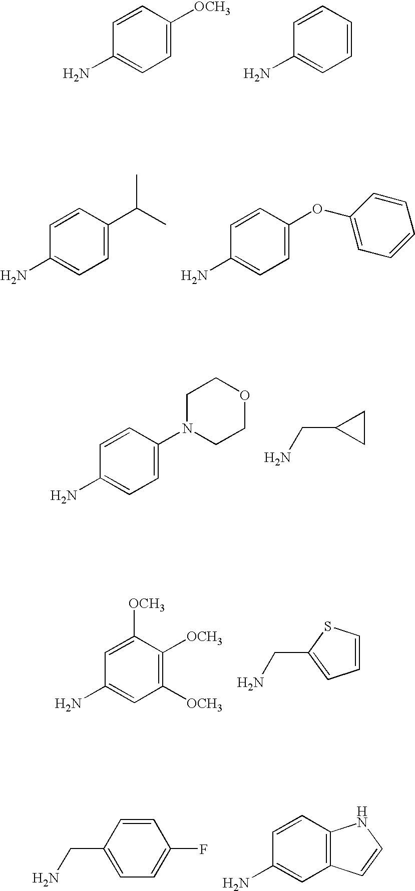
and wherein R₂ is selected from the group consisting of the following structures:

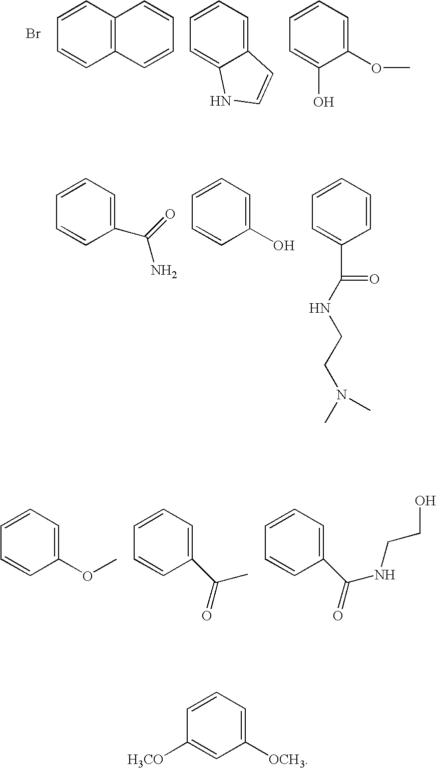

4. A method of treating a proliferative disease comprising administering to a mammal in need thereof, a therapeutically effective amount of a composition according to claim 1.

5. The method of claim 4 wherein the mammal is a human.

6. The method of claim 4 wherein the proliferative disease is cancer.

7. A method of treating a proliferative disease comprising administering to a mammal in need thereof, a therapeutically effective amount of a composition according to claim 2.

8. The method of claim 7 wherein the mammal is a human.

9. The method of claim 7 wherein the proliferative disease is cancer.

10. A method of treating a proliferative disease comprising administering to a mammal in need thereof, a therapeutically effective amount of a composition according to claim 3.

11. The method of claim 10 wherein the mammal is a human.

12. The method of claim 10 wherein the proliferative disease is cancer.
